(19) United States
(12) Patent Application Publication
(10) Pub. No.: US 2005/0059605 A1
(43) Pub. Date: Mar. 17, 2005

(54) Chemically modified metabolites of regulatory peptides and methods of producing and using same

(76) Inventors: Krishna Peri, St-Laurent (CA); Abdelkrim Habi, Dollard des Ormeaux (CA); Denis Gravel, St-Lambert (CA)

Correspondence Address: FULBRIGHT & JAWORSKI L.L.P., 600 CONGRESS AVE., SUITE 2400, AUSTIN, TX, 78701, US

(21) Appl. No.: 10/768,974

(22) Filed: Jan. 30, 2004

Related U.S. Application Data
(60) Provisional application No. 60/443,860, filed on Jan. 31, 2003.

(51) Int. Cl. A61K 38/08; C07K 7/06

(52) U.S. Cl. 514/17; 530/330; 514/18

(57) ABSTRACT

The present invention relates to a peptide of Formula I, or a pharmaceutically acceptable salt thereof:
X—P Formula I wherein: P is a DPPIV peptide metabolite of regulatory peptides obtained by cleavage of the two N-terminal amino acids; and
X is defined by Formula II: 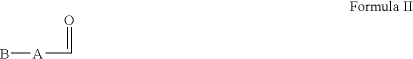 wherein:
A is selected from the group consisting of C₁-C₁₀ alkylene, C₂-C₁₀ alkenylene, C₂-C₁₀ alkynylene, C₁-C₁₀ heteroalkylene, C₂-C₁₀ heteroalkenylene, C₂-C₁₀ heteroalkynylene and phenyl; and
B is selected from the group consisting of aryl, substituted aryl, heteroaryl, substituted heteroaryl and C₃-C₇ cycloalkyl.

### FIELD OF THE INVENTION

This application claims the priority of U.S. Provisional Patent Application No. 60/443,860, filed Jan. 31, 2003, the entire disclosure of which is specifically incorporated herein by reference.

The present invention relates to chemically modified metabolites of regulatory peptides. The present invention also relates to methods of producing and using the chemically modified metabolites. More specifically, the present invention relates to conferring biological activity to metabolites of regulatory peptides by the covalent coupling of small molecules.

### BACKGROUND OF THE INVENTION

Regulatory peptides are diverse in view of the plethora of neurological, immunomodulatory, anti-/pro-inflammatory, and gastrointestinal, metabolic functions they mediate in the body. A subset of these peptides (Table 1) is metabolized by dipeptidyl peptidases, members of the prolyl oligopeptidase/serine protease family.

Dipeptidyl-peptidase IV (DPPIV, EC 3.4.14.5, CD26), also designated CD26, is an extracellular membrane-bound enzyme expressed on the surface of several cell types, in particular CD4 and T-cells, as well as on kidney, placenta, blood plasma, liver, and intestinal cells. On T-cells, DPPIV has been shown to be identical to the antigen CD26. CD26 is expressed on a fraction of resting T-cells at low density, but is strongly up-regulated following T-cell activation (Gorrell, M. D. et al. 2001; Scand. J. Immunol. 54(3): 249-264).

Human serum contains abundant amounts of soluble CD26, which is responsible for serum DPPIV activity. Serum DPPIV is a 250 kDa homodimer, inhibited by Diprotin A and heavy metals (Shibuya-Saruta, H. et al. 1996; J. Clin. Lab. Anal. 10(6): 435-40). Recent results have indicated that CD26 is a multifunctional molecule that may have an important functional role in T-cells, as well as in overall immune system modulation. CD26 is associated with other receptors of immunological significance found on the cell surface such as protein tyrosine phosphatase CD45 and adenosine deaminase (ADA).

Another important function of DPPIV is to truncate several bioactive peptides and proteins such as those listed in Table 1 by two N-terminal amino acids, thus inactivating or revealing new bioactivity for the truncated peptides (De Meester, I. et al. 2000; Cellular peptidases in immune functions and diseases 2; Eds. Langner and Ansorge; Kluver Academic/Plenum Press).

DPPIV prefers peptides having the X-Ala or X-Pro N-terminal motif. It is therefore hypothesized that DPPIV plays a role in the inactivation of regulatory peptides such as GHRH, GLP-1, GLP-2, GIP and glucagon, and may thus exert metabolic control. In fact, it has been shown that DPPIV-null mice exhibit improved glucose tolerance and increased secretion of GLP-1 (Marguet, D. et al. 2000; Proc. Natl. Acad. Sci. USA. 97(12): 6874-79).

TABLE 1

DPPIV substrates, their metabolites and their functions.

| | | |
|---|---|---|
| Substrate | Sequence | Comment |

| | | |
|---|---|---|
| Substance P | ArgProLeuProGlnGluPhePheGlyLeuMet- | Arg Pro and Leu Pro cleaved; |
| SP | amide | SP(5-11) increased activity |
| Beta | TyrProPheProGly | Potent opiold-like; isolated |
| Casomorphin-5 | | from bovine milk; inactivation |
| | | by cleavage |
| Endomorphin-2 | TyrProPhePhe-NH₂ | High affinity mu opioid |
| | | receptor ligand; inactivated by |
| | | cleavage |
| Procolipase | 100 aa peptide (X1-Pro-X2-Pro-Arg . . .) | Cleavage results in colipase |
| | | and enterostatin |
| Enterostatin | ValProAspProArg . . . | Enterostatin i.p injections |
| | | produce low fat intake; |
| | | chronic treatment reduced |
| | | body weight and body fat |
| Neuropeptide Y | Tyr-Pro-Ser-Lys-Pro-Asp-Asn-Pro . . . | Tyr Pro cleavage liberates |
| | | more selective peptide; NPY |
| | | stimulates food intake, |
| | | lipogenesis, |
| | | anxiolysis/sedation. |
| Peptide YY | Tyr-Pro-Ile-Lys-Pro-Glu-Ala-Pro . . . | PYY localized in endocrine |
| | | cells of gastric mucosa, |
| | | inhibits pancreatic secretion, |
| | | vasoconstriction, inhibits |
| | | jejunal and colonic motility. |
| | | Cleaved metabolite (3-36) |
| | | suppresses food intake in a |
| | | receptor (Y2) selective |
| | | manner. |
| Glucagon | TyrAla, HisAla or HisSer at the N- | GLP-1: stimulates insulin |
| superfamily: | terminus | secretion in glucose- |
| 1. Glucagon | | dependent manner. (3-37) or |
| 2. GLP-1 | | (3-36) amide lost incretin |
| 3. GLP-2 | | activity. |
| 4. VIP | | GLP-2: intestinal growth |
| 5. GIP | | factor activity. Metabolite is |
| 6. GHRH | | inactive. |
| | | GIP: insulin secretogogue; |
| | | Metabolite is inactive. |
| | | GHRH: pulsatile secretion of |
| | | GH; metabolite is inactive |
| | | VIP: several functions in |
| | | peripheral and CNS; |
| Chromogranin A | Share same N-terminus but are 431, 76 | Chromogranin A; acidic |
| Vasostatin I & II | & 113 residues respectively. | protein; distributed in |
| | | secretory granules of |
| | | endocrine and |
| | | neuroendocrine tissues, |
| | | precursor of vasostatin I & II |
| | | Vasostatin I: suppress ET1- |
| | | induced contractions of blood |
| | | vessels |
| | | Vasostatin II: inhibits PTH |
| | | secretion stimulated by low |
| | | Ca²⁺ |
| Calcitonin gene | Conserved Pro in 2nd position. | May be involved in CT, |
| family | | CGRP generation |
| Procalcitonin, | | |
| proNCT & | | |
| proCGRP | | |
| Chemokine | SerAlaLysGluLeuArgCysGlnCys . . . | Chemokines involved in |
| family | GlyProValSerAlaValLeuThrGluLeu . . . | immune cell recruitment |
| | GluAlaGluGluAspGlyAspLeuGlnCys . . . | and/or inflammation |
| CXC-group | ValProLeuSerArgThrValArgCysThrCys . . . | |
| IL8 | ThrProValValArgLysGlyArgCysSerCys . . . | |
| GCP-2 | LysProValSerLeuSerTyrArgCysProCys . . . | |
| PF4 | AlaProLeuAlaThrGluLeuArgCysGlnCys . . . | |
| IP-10 | PheProMetPheLysLysGlyArgCysLeuCys . . . | |
| MIG | | |
| SDF-1α | | |
| GRO-α | | |
| I-TAC | | |
| CC-group | SerProTyrSerSerAspThrThrProCys . . . | RANTES: Truncation reduced |
| RANTES | AlaProLeuAlaAlaAspThrProThrAlaCys . . . | signaling; poor |
| LD78 | AlaProMetGlySerAspProProThrAlaCys . . . | chemoattractant to |
| MIP-1α | GlnProAspAlaIleAsnAlaProValThrCys . . . | monocytes and neutrophiles |
| MCP-1 | GlnProSerAspValSerIleProIleThrCys . . . | SDF-1: Truncation reduced |
| MCP-2 | GlnProValGlyIleAsnSerThrThrCys . . . | chemo-attractant and antiviral |
| MCP-3 | GlnProAspAlaLeuAspValProSerThrCys . . . | activity |
| MCP-4 | GlyProAlaSerValProThrThrCys . . . | Eotaxin: Truncation reduced |
| Eotaxin | GlyProTyrGlyAlaAsnMetGluAspSerVal- | eosinophile attractant activity; |
| | Cys . . . | inhibitor of CCR3 |
| MDC | | |

Pharmacological inhibition of DPPIV leads to increased glucose control in normal and diabetic mice (Ahren, B. et al. 2000; Eur. J. Pharmacol. 404(1-2): 239-45; O' Hart; F. P. et al. 2000; J. Endocrinol. 165(3): 639-48). Increased peptide levels of GIP and GLP-2 were also observed in animals treated with DPPIV inhibitors (Hartmann, B. et al. 2000; Eur. J. Endocrinol. 141(11): 4013-20; Deacon, C. F. et al. 2001; Diabetes 50(7): 1588-97). DPPIV resistant analogues of regulatory peptides are thus capable of providing suitable drugs for different medical conditions.

DPPIV metabolites, following N-terminal dipeptide cleavage, circulate in the blood for much longer periods of time than the parent peptide. For example, the plasma half life of active GLP-1 is <5 min., whereas the metabolic clearance rate of the metabolite requires about 12-13 min. (Hoist, J. J. 1994; Gastroenterology 107: 1848-1855). Similarly GHRH, GIP and GLP-2 display short half-lives in circulation (2-4 min.). The metabolites have no observed biological activity (e.g. GHRH), no weak agonist or antagonist activity (e.g. GLP-1), nor any new biological property (NPY, PYY, RANTES etc.).

There thus remains a need for chemically modified metabolites of regulatory peptides having biological activity and potency similar to the native peptides.

The present invention seeks to meet these and other needs.

The present description refers to a number of documents, the content of which is herein incorporated by reference in their entirety.

### SUMMARY OF THE INVENTION

The present invention relates to conferring biological activity to metabolites of regulatory peptides, by the covalent coupling of molecules selected from a discrete set of arylalkyl groups. A structure-activity relationship (SAR) was found defining the general structure of a pharmacophore that could be coupled to the N-terminus of DPPIV metabolites, thus conferring biological activity to the metabolites. The peptide metabolites are obtained from the native peptides by cleavage of the two N-terminal amino acids by dipeptidyl peptidases.

The present invention relates to a peptide of Formula I, or a pharmaceutically acceptable salt thereof:
X—P Formula I
wherein:
P is a DPPIV peptide metabolite of regulatory peptides obtained by cleavage of the two N-terminal amino acids; and
X is defined by Formula II: 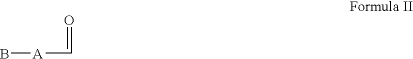
wherein A is selected from the group consisting of C₁-C₁₀ alkylene, C₂-C₁₀ alkenylene, C₂-C₁₀ alkynylene, C₁-C₁₀ heteroalkylene, C₂-C₁₀ heteroalkenylene, C₂-C₁₀ heteroalkynylene and phenyl; and
B is selected from the group consisting of aryl, substituted aryl, heteroaryl, substituted heteroaryl and C₃-C₇ cycloalkyl.

The present invention relates to a composition comprising a therapeutically effective amount of a peptide as defined herein, in association with at least one constituent selected from the group consisting of pharmaceutically acceptable carrier, diluents or excipients.

The present invention relates to a composition comprising a prophylactically effective amount of a peptide as defined herein, in association with at least one constituent selected from the group consisting of pharmaceutically acceptable carrier, diluents or excipients.

In one embodiment, when the regulatory peptide is GLP-1, the present invention relates to a method for treating or preventing a disease or condition associated with a disorder of glucose metabolism. The invention, in a further embodiment, relates to a prevention (e.q. prophylaxis) of a disease or condition associated with a disorder of glucose metabolism. Non-limiting examples of glucose disorder include: diabetes mellitus of Type I or Type II, insulin resistance, weight disorders and diseases or conditions associated thereto, wherein such weight disorders or associated conditions include obesity, overweight-associated conditions, satiety deregulation, reduced plasma insulin levels, increased blood glucose levels, or reduced pancreatic beta cell mass.

The present invention also relates to methods of synthesizing the peptides of Formula 1 (X—P).

In addition, the present invention relates to methods of testing the peptides of Formula I in order to compare their biological activities with those of their parent peptides.

Further scope and applicability will become apparent from the detailed description given hereinafter. It should be understood however, that this detailed description, while indicating preferred embodiments of the invention, is given by way of illustration only, since various changes and modifications within the spirit and scope of the invention will become apparent to those skilled in the art.

### BRIEF DESCRIPTION OF THE DRAWINGS

FIG. 1 shows second messenger (cAMP) synthesis in rat insulinoma cells (RINm5F) produced by the GLP-1 analogues of the present invention. Several compounds were observed as having comparable Eₘₐₓ and EC₅₀ values to Exendin-4 (EX-4), 234 (GLP-1 [7-36]amide) and 260 (Gly8 GLP-1 [7-36] amide);

FIG. 2 shows the insulin secretion stimulated by GLP-1 analogues of the present invention in response to the intraperitoneal glucose tolerance test (IPGTT). Insulin levels (30, 60 and 90 minutes following glucose challenge) are averaged. Taking the average insulin level of 234 (GLP-1 [7-36] amide) and 260 (Gly8 GLP-1 [7-36] amide) as 1.0, the fold increase in insulin produced by the compounds are calculated and presented as a bar graph. The horizontal line represents the average insulin level produced by 234 and 260. The compounds that produced average insulin levels at or above the line are considered to be equally or more potent than compounds 234 and 260.

FIG. 3 shows the effects of glucagon and analogues on freshly isolated hepatocyte cAMP production (n=2; mean±SEM). Replacement of His-Ser dipeptide by a synthetic mimic in compound 361 elicited significantly higher intracellular cAMP levels as compared to analogue 357 lacking the His-Ser dipeptide (negligible response) or glucagon itself.

FIG. 4 shows dose response curves of GLP-1 analogs on cAMP production in RINm5F cells (n=3; mean±SEM; legend presented as drug EC₅₀). Analogue 277 is GLP-1 (9-36) amide, and analogue 288 contains a synthetic mimic in place of N-terminal His-Ala dipeptide.

FIG. 5 shows the effects of 25 μg/mice (500 μg/kg) subcutaneous injections of analogue 288, 234 (GLP-1 (7-36)NH₂), Exendin-4 or saline, on glucose levels following 30 minutes of feeding subsequent to overnight fasting in C57BL/ks db/db mice (data is shown as mean±SEM). Compared to native GLP-1, analogue 288 produced a more significant hypoglycemic response.

FIG. 6 shows cAMP production stimulated by GRF analogues (10⁻⁶ M) in whole anterior pituitary culture. Analogue 358 produced a significantly greater cAMP response than analogue 356. For comparison, a DPPIV resistant analogue of GRF (analogue 280) was shown.

Other objects, advantages and features of the present invention will become more apparent upon reading the following non-restrictive description of preferred embodiments with reference to the accompanying drawings, which is exemplary and should not be interpreted as limiting the scope of the present invention.

### DETAILED DESCRIPTION OF THE INVENTION

Definitions:

As used herein, the term “pharmaceutical excipient” means any material used in the formulation of a medicament that is not an active pharmaceutical ingredient. Non-limiting examples of pharmaceutical excipients include binders, fillers, disintegrants, diluents, coating agents, flow enhancers and lubricants.

As used herein, the term “alkylene” refers to a straight or branched saturated acyclic carbon chain comprising from 1 to 10 carbon atoms, preferably 3 to 8 carbon atoms, and more preferably 5 carbon atoms.

As used herein, the term “alkenylene” refers to a straight or branched unsaturated acyclic carbon chain comprising from 2 to 10 carbon atoms, preferably 3 to 8 carbon atoms, and more preferably 5 carbon atoms.

As used herein, the term “alkynylene” refers to a straight or branched unsaturated acyclic carbon chain comprising from 2 to 10 carbon atoms, preferably 3 to 8 carbon atoms, and more preferably 5 carbon atoms.

As used herein, the term “heteroalkylene” refers to a straight or branched saturated acyclic carbon chain as previously defined, wherein one or more of the carbon atoms have been substituted with heteroatoms selected from the group consisting of oxygen, nitrogen, sulfur and combinations thereof, and/or with functional groups selected from the group consisting of carbonyl, sulfonyl, and combinations thereof, and wherein one or more of the heteroatoms may be flanked by one or more of the functional groups.

As used herein, the term “heteroalkenylene” refers to a straight or branched unsaturated acyclic carbon chain as previously defined, wherein one or more of the carbon atoms have been substituted with heteroatoms selected from the group consisting of oxygen, nitrogen, sulfur, and combinations thereof, and/or with functional groups selected from the group consisting of carbonyl, sulfonyl, and combinations thereof, and wherein one or more of the heteroatoms may be flanked by one or more of the functional groups.

As used herein, the term “heteroalkynylene” refers to a straight or branched unsaturated acyclic carbon chain as previously defined, wherein one or more of the carbon atoms have been substituted with heteroatoms selected from the group consisting of oxygen, nitrogen, sulfur and combinations thereof, and/or with functional groups selected from the group consisting of carbonyl, sulfonyl, and combinations thereof, and wherein one or more of the heteroatoms may be flanked by one or more of the functional groups.

As used herein, the term “aryl” refers to phenyl, 1-naphtyl, 2-naphtyl, or biphenyl.

As used herein, the term “substituted aryl” refers to phenyl, 1-naphthyl, 2-naphthyl, or biphenyl having a substituent selected from the group consisting of lower alkyl, lower alkoxy, lower alkylthio, halo, hydroxy, trifluoromethyl, amino, —NH(lower alkyl), and —N(lower alkyl)₂, or refers to di- and tri-substituted phenyl, 1-naphthyl, 2-naphthyl, or biphenyl, wherein the substituents are selected from the group consisting of methyl, methoxy, methylthio, halo, hydroxy, and amino.

As used herein, the term “heteroaryl” refers to a heterocyclic aromatic ring system containing one or more heteroatoms selected from nitrogen, oxygen and sulfur. Non-limiting examples include furanyl, thiophenyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl (thianaphthenyl), indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, carbazolyl, azepinyl, diazepinyl, and acridinyl. Heteroaryl ring systems may be substituted at an available carbon atom by a lower alkyl, halo, hydroxy, benzyl, or cyclohexylmethyl group. Furthermore, the heteroaryl ring systems may be substituted at an available N-atom by an N-protecting group (Green, T. W.; Wuts, P. G. M.: “Protective Groups in Organic Synthesis”, 3^{rd} Edition, John Wiley & Sons, NY, 1999, pp 494-653).

As used herein, the term “lower alkyl” refers to straight or branched chain radicals having 1 to 4 carbon atoms.

As used herein, the terms “alkoxy” and “alkylthio” refer to alkyl groups attached to an oxygen or a sulfur atom, respectively.

As used herein, the term “cycloalkyl” refers to saturated rings of 3 to 7 carbons atoms.

As used herein, the term “heterocycloalkyl” refers to a saturated 3 to 8-membered ring containing one or more heteroatoms selected from nitrogen, oxygen and sulfur. Representative examples are pyrrolidyl, piperidyl, piperazinyl, morpholinyl, thiomorpholinyl, aziridinyl, tetrahydrofuranyl and the like.

Non-limiting examples of A as defined herein include propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), —O—CH₂CH₂—S—CH₂—, —CH₂C₆H₄—, —CH₂—CO—N H—CH₂CH₂—, —C(O)—(CH₂)₄—, —CH₂CH₂N HC(O)CH₂—, —CH₂CH₂C(O)NHCH₂CH₂CH₂CH₂—, —CH₂CH₂CH₂N HC(O)CH₂—, —SO₂—N H—CH₂CH₂CH₂—, —C(O)NHCH₂CH₂CH₂CH₂—, —C(O)NHCH₂CH₂CH₂—, (trans) —NHC(O)CH═CH—, and (cis) —NHC(O)CH═CH.

Further examples of A, contemplated as being within the scope of the present invention, are those alkylene, alkenylene, alkynylene, heteroalkylene, heteroalkenylene, heteroalkynylene chains as previously defined, further comprising an aryl or heteroaryl moiety, either as a substituent or as a part of the chain.

The amino acids, as described herein, are identified by the conventional three-letter abbreviations as indicated below in Table 2, which are as generally accepted in the peptide art as recommended by the IUPAC-IUB commission in biochemical nomenclature:

TABLE 2
Amino acid codes

| | 3-letter | 1-letter | | 3-letter | 1-letter |
|---|---|---|---|---|---|
| Name | code | code | Name | code | code |
| Alanine | Ala | A | Leucine | Leu | L |
| Arginine | Arg | R | Lysine | Lys | K |
| Asparagine | Asn | N | Methionine | Met | M |
| Aspartic | Asp | D | Phenylalanine | Phe | F |
| Cysteine | Cys | C | Proline | Pro | P |
| Glutamic acid | Glu | E | Serine | Ser | S |
| Glutamine | Gln | Q | Threonine | Thr | T |
| Glycine | Gly | G | Tryptophan | Trp | W |
| Histidine | His | H | Tyrosine | Tyr | Y |
| Isoleucine | Ile | I | Valine | Val | V |

The peptide sequences as described herein, are written in accordance to the generally accepted convention, whereby the N-terminal amino acid is on the left hand side and the C-terminal amino acid is on the right hand side.

In a broad sense, the present invention relates to sequences of peptide metabolites, produced by the action of serine protease/oligoprolyl protease/dipeptidyl protease members, and more preferably DPPIV on the native peptides. Moreover, the present invention relates to peptide metabolites (“P”), produced by the actions of dipeptidyl peptidases, more specifically DPPIV, on regulatory peptides, preferably those listed in Table 3. The peptide metabolites that have lost the N-terminal dipeptide are deficient in biological activity and potency, as compared to the native peptide.

TABLE 3

DPPIV metabolites (“P”) of metabolic regulatory peptides.

REGULATORY

| | |
|---|---|
| PEPTIDE | SEQUENCE OF DPPIV METABOLITES “P” (N to C) |

| | |
|---|---|
| Glucagon like peptide- | Glu-Gly-Thr-Phe-Thr-Ser-Asp-Val-Ser-Ser-Tyr-Leu-Glu-Gly-Gln- |
| 1 (GLP-1) | Ala-Ala-Lys-Glu-Phe-Ile-Ala-Trp-Leu-Val-Lys-Gly-Arg-X (X = NH₂ or |
| | Gly-OH) |
| Glucagon like peptide- | Asp-Gly-Ser-Phe-Ser-Asp-Glu-Met-Asn-Thr-Ile-Leu-Asp-Asn-Leu- |
| 2 (GLP-2) | Ala-Ala-Arg-Asp-Phe-Ile-Asn-Trp-Leu-Ile-Gln-Thr-Lys-Ile-Thr-Asp- |
| | Arg |
| Growth hormone | Asp-Ala-Ile-Phe-Thr-Asn-Ser-Tyr-Arg-Lys-Val-Leu-Gly-Gln-Leu- |
| releasing hormone | Ser-Ala-Arg-Lys-Leu-Leu-Gln-Asp-Ile-Met-Ser-Arg-Gln-Gln-Gly- |
| (GHRH) | Glu-Ser-Asn-Gln-Glu-Arg-Gly-Ala-Arg-Ala-Arg-Leu-NH₂ |
| Vasoactive intestinal | Asp-Ala-Val-Phe-Thr-Asp-Asn-Tyr-Thr-Arg-Leu-Arg-Lys-Gln-Met- |
| peptide (VIP) | Ala-Val-Lys-Lys-Tyr-Leu-Asn-Ser-Ile-Leu-Asn-NH₂ |
| Glucose-dependent | Glu-Gly-Thr-Phe-Ile-Ser-Asp-Tyr-Ser-Ile-Ala-Met-Asp-Lys-Ile-His- |
| insulinotropic peptide | Gln-Gln-Asp-Phe-Val-Asn-Trp-Leu-Leu-Ala-Gln-Lys-Gly-Lys-Lys- |
| (GIP) | Asn-Asp-Trp-Lys-His-Asn-Ile-Thr-Gln |
| Glucagon | Gln-Gly-Thr-Phe-Thr-Ser-Asp-Tyr-Ser-Lys-Tyr-Leu-Asp-Ser-Arg- |
| | Arg-Ala-Gln-Asp-Phe-Val-Gln-Trp-Leu-Met-Asn-Thr |
| Neuropeptide Y | Ser-Lys-Pro-Asp-Asn-Pro-Gly-Glu-Asp-Ala-Pro-Ala-Glu-Asp-Met- |
| | Ala-Arg-Tyr-Tyr-Ser-Ala-Leu-Arg-His-Tyr-Ile-Asn-Leu-Ile-Thr-Arg- |
| | Gln-Arg-Tyr |
| Peptide YY | Ile-Lys-Pro-Glu-Ala-Pro-Gly-Glu-Asp-Ala-Ser-Pro-Glu-Glu-Leu-Asn- |
| | Arg-Tyr-Tyr-Ala-Ser-Leu-Arg-His-Tyr-Leu-Asn-Leu-Val-Thr-Arg- |
| | Gln-Arg-Tyr-NH₂ |
| Gastrin Releasing | Leu-Pro-Ala-Gly-Gly-Gly-Thr-Val-Leu-Thr-Lys-Met-Tyr-Pro-Arg-Gly- |
| Peptide (GRP) | Asn-His-Trp-Ala-Val-Gly-His-Leu-Met-NH₂ |

In a preferred embodiment of the present invention, the DPPIV substrates are selected from the group of regulatory peptides consisting of, but not limited to, Growth Hormone Releasing Factor (GRF) (1-29), Glucagon-Like Peptide 1 (7-37) amide, human GLP-1, GLP-2, human peptide YY, GIP, Peptide YY, Neuropeptide Y, Eotaxin and Substance P.

In the course of testing a variety of molecules capable of replacing the N-terminal dipeptide removed from the regulatory peptides listed in Table 1 as the results of the action of DPPIV, and capable of conferring biological activity representative of the native peptide, it was unexpectantly discovered that these molecules generally posses a generic structure.

In a further preferred embodiment, the present invention relates to chemically modified metabolites of regulatory peptides wherein the N-terminal dipeptide is replaced by a small molecule, conferring biological activity and potency representative of the native peptide.

In yet a further embodiment, the present invention relates to a peptide of Formula I, or a pharmaceutically acceptable salt thereof:
X—P Formula I
wherein:
P is a DPPIV peptide metabolite of regulatory peptides obtained by cleavage of the two N-terminal amino acids; and
X is defined by Formula II: 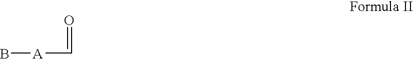
wherein A is selected from the group consisting of C₁-C₁₀ alkylene, C₂-C₁₀ alkenylene, C₂-C₁₀ alkynylene, C₁-C₁₀ heteroalkylene, C₂-C₁₀ heteroalkenylene, C₂-C₁₀ heteroalkynylene and phenyl; and
B is selected from the group consisting of aryl, substituted aryl, heteroaryl, substituted heteroaryl and C₃-C₇ cycloalkyl.

In yet a further embodiment, the present invention relates to methods of synthesizing the peptides of formula I (X—P).

In yet another embodiment, the present invention relates to a composition comprising a therapeutically effective amount of a peptide as defined herein, in association with at least one constituent selected from the group consisting of pharmaceutically acceptable carrier, diluents or excipients.

In yet another embodiment, the present invention relates to a composition comprising a prophylactically effective amount of a peptide as defined herein, in association with at least one constituent selected from the group consisting of pharmaceutically acceptable carrier, diluents or excipients.

In yet a further embodiment, the present invention relates to methods of testing the peptides of Formula I in order to compare their biological activities with those of their parent peptides.

In yet a further preferred embodiment, P is a DPPIV peptide metabolite of regulatory peptides. In a more preferred embodiment, P is a DPPIV peptide metabolite of regulatory peptides, non-limiting examples of which are listed in Table 3.

Regulatory peptides such as those listed in Table 3 can be modified by known methods in the art including amidation of the terminal carboxyl group, substitution of one or more amino acids with synthetic amino acids, modification of one or more amino acids with saturated or unsaturated acyl chains ranging from 10 to 20 carbons (C₁₀-C₂₀), cyclization and rigidification of the secondary structure via lactam bridges, or PEGylation using PEG groups ranging from 2-20 kDa. These modifications result in peptides having higher potency, higher solubility, enhanced plasma half life due to their resistance to proteases including DPPIV, increased peptide stability owing to resistance to oxidation, deamidation and other chemical changes that occur upon storage. It is intended that the peptide metabolite “P” includes the peptide sequences listed in Table 3 (native regulatory peptides following N-terminal dipeptide cleavage), but also includes those peptide sequences modified according to the description given above.

In yet a further preferred embodiment of the present invention, X is selected from the group of structures listed in Table 4.

TABLE 4
Structures of “X”

| | |
|---|---|
| 1 | 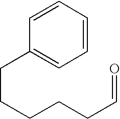 |
| 2 | 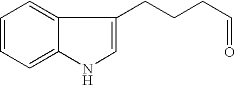 |
| 3 | 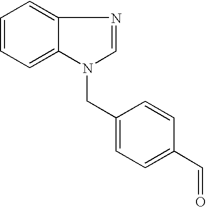 |
| 4 | 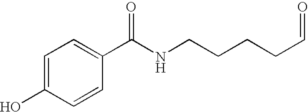 |
| 5 | 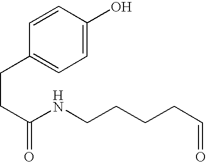 |
| 6 | 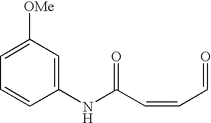 |
| 7 | 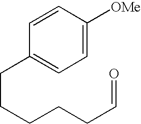 |
| 8 | 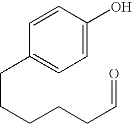 |
| 9 | 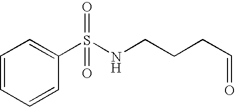 |
| 10 | 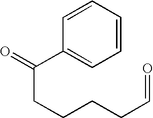 |
| 11 | 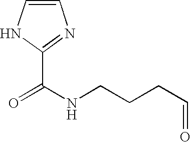 |
| 12 | 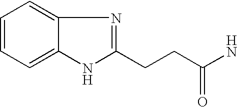 |
| 13 | 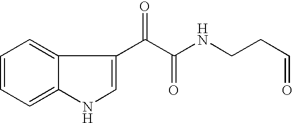 |
| 14 | 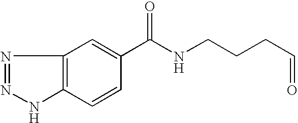 |
| 15 | 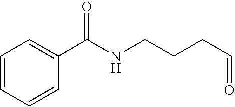 |
| 16 | 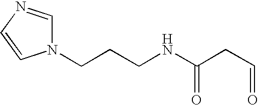 |
| 17 | 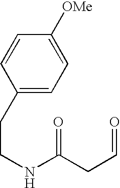 |
| 18 | 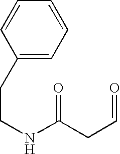 |
| 19 | 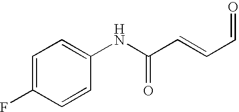 |
| 20 | 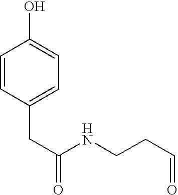 |
| 21 | 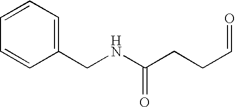 |
| 22 | 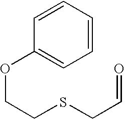 |
| 23 | 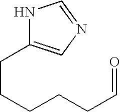 |
| 24 | 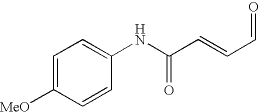 |
| 25 | 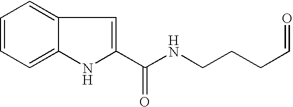 |
| 26 | 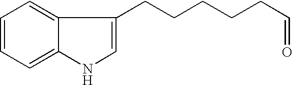 |
| 27 | 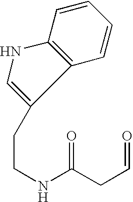 |
| 28 | 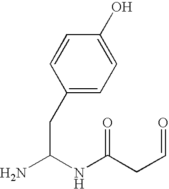 |
| 29 | 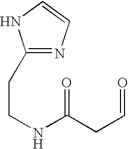 |
| 30 | 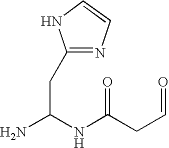 |
| 31 | 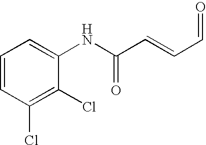 |
| 32 | 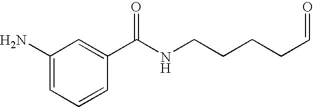 |
| 33 | 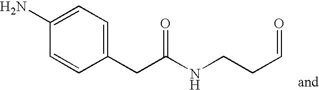 |
| 34 | 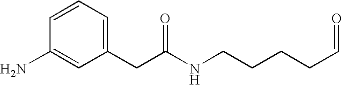 |

In a first embodiment of the present invention, “P” is Glucagon Like Peptide-1 metabolite (GLP-1) (9-37), (9-36) amide, (9-39) amide or (9-44) amide, having the sequences:

| | |
|---|---|
| GLP-1 (9-44) NH₂: | Glu-Gly-Thr-Phe-Thr-Ser-Asp-Val-Ser-Ser- |
| | Tyr-Leu-Glu-Gly-Gln-Ala-Ala-Lys-Glu-Phe- |
| | Ile-Ala-Trp-Leu-Val-Lys-Gly-Arg-Gly-Arg- |
| | Arg-Asp-Phe-Pro-Glu-Glu-NH₂. |
| GLP-1 (9-39) NH₂: | Glu-Gly-Thr-Phe-Thr-Ser-Asp-Val-Ser-Ser- |
| | Tyr-Leu-Glu-Gly-Gln-Ala-Ala-Lys-Glu-Phe- |
| | Ile-Ala-Trp-Leu-Val-Lys-Gly-Arg-Gly-Arg- |
| | Arg-NH₂. |
| GLP-1 (9-37) OH: | Glu-Gly-Thr-Phe-Thr-Ser-Asp-Val-Ser-Ser- |
| | Tyr-Leu-Glu-Gly-Gln-Ala-Ala-Lys-Glu-Phe- |
| | Ile-Ala-Trp-Leu-Val-Lys-Gly-Arg-Gly-OH. |
| GLP-1 (9-36) NH₂: | Glu-Gly-Thr-Phe-Thr-Ser-Asp-Val-Ser-Ser- |
| | Tyr-Leu-Glu-Gly-Gln-Ala-Ala-Lys-Glu-Phe- |
| | Ile-Ala-Trp-Leu-Val-Lys-Gly-Arg-NH₂. |

In a second embodiment of the present invention, “P” is Glucagon Like Peptide-2 GLP-2 (3-34) or GLP-2 (3-33) metabolite having the sequences:

| | |
|---|---|
| GLP-2 (3-34): | Asp-Gly-Ser-Phe-Ser-Asp-Glu-Met-Asn-Thr- |
| | Ile-Leu-Asp-Asn-Leu-Ala-Ala-Arg-Asp-Phe- |
| | Ile-Asn-Trp-Leu-Ile-Gln-Thr-Lys-Ile-Thr- |
| | Asp-Arg-NH₂ |
| GLP-2 (3-33): | Asp-Gly-Ser-Phe-Ser-Asp-Glu-Met-Asn-Thr- |
| | Ile-Leu-Asp-Asn-Leu-Ala-Ala-Arg-Asp-Phe- |
| | Ile-Asn-Trp-Leu-Ile-Gln-Thr-Lys-Ile-Thr- |
| | Asp-NH₂ |

In a third embodiment of the present invention, “P” is Growth Hormone Releasing Factor GRF (3-44) NH₂ or GRF (3-29) NH₂ metabolite having the sequences:

| | |
|---|---|
| GRF (3-44) NH₂: | Asp-Ala-Ile-Phe-Thr-Asn-Ser-Tyr-Arg-Lys- |
| | Val-Leu-Gly-Gln-Leu-Ser-Ala-Arg-Lys-Leu- |
| | Leu-Gln-Asp-Ile-Met-Ser-Arg-Gln-Gln-Gly- |
| | Glu-Ser-Asn-Gln-Glu-Arg-Gly-Ala-Arg-Ala- |
| | Arg-Leu-NH₂. |
| GRF (3-29) NH₂: | Asp-Ala-Ile-Phe-Thr-Asn-Ser-Tyr-Arg-Lys- |
| | Val-Leu-Gly-Gln-Leu-Ser-Ala-Arg-Lys-Leu- |
| | Leu-Gln-Asp-Ile-Met-Ser-Arg-NH₂. |

In a fourth embodiment of the present invention, “P” is vasoactive intestinal peptide (VIP) (3-28) NH₂ metabolite having the sequence:

Asp-Ala-Val-Phe-Thr-Asp-Asn-Tyr-Thr-Arg-Leu-Arg-
Lys-Gln-Met-Ala-Val-Lys-Lys-Tyr-Leu-Asn-Ser-Ile-
Leu-Asn-NH₂.

In a fifth embodiment of the present invention, “P” is Glucose-Dependent Insulinotropic Peptide GIP (3-42) NH₂ or GIP (3-30) NH₂ metabolite having the sequences:

| | |
|---|---|
| GIP (3-42) NH₂: | Glu-Gly-Thr-Phe-Ile-Ser-Asp-Tyr-Ser-Ile-Ala-Met-Asp-Lys-Ile-His-Gln-Gln-Asp-Phe-Val- |
| | Asn-Trp-Leu-Leu-Ala-Gln-Lys-Gly-Lys-Lys-Asn-Asp-Trp-Lys-His-Asn-Ile-Thr-Gln-NH₂. |
| GIP (3-30) NH₂: | Glu-Gly-Thr-Phe-Ile-Ser-Asp-Tyr-Ser-Ile-Ala-Met-Asp-Lys-Ile-His-Gln-Gln-Asp-Phe-Val- |
| | Asn-Trp-Leu-Leu-Ala-Gln-Lys-NH₂. |

In a sixth embodiment of the present invention, “P” is Glucagon (3-29) NH₂ metabolite having the sequence:

Gln-Gly-Thr-Phe-Thr-Ser-Asp-Tyr-Ser-Lys-Tyr-Leu-
Asp-Ser-Arg-Arg-Ala-Gln-Asp-Phe-Val-Gln-Trp-Leu-
Met-Asn-Thr.

In a seventh embodiment of the present invention, “P” is neuropeptide Y (3-36) NH₂ metabolite having the sequence:

Ser-Lys-Pro-Asp-Asn-Pro-Gly-Glu-Asp-Ala-Pro-Ala-
Glu-Asp-Met-Ala-Arg-Tyr-Tyr-Ser-Ala-Leu-Arg-His-
Tyr-Ile-Asn-Leu-Ile-Thr-Arg-Gln-Arg-Tyr.

In an eighth embodiment of the present invention, “P” is peptide YY (3-29) NH₂ metabolite having the sequence:

Ile-Lys-Pro-Glu-Ala-Pro-Gly-Glu-Asp-Ala-Ser-Pro-
Glu-Glu-Leu-Asn-Arg-Tyr-Tyr-Ala-Ser-Leu-Arg-His-
Tyr-Leu-Asn-Leu-Val-Thr-Arg-Gln-Arg-Tyr-NH₂.

In a ninth embodiment of the present invention, “P” is Gastrin Releasing Peptide (GRP) (3-27) NH₂ metabolite having the sequence:

Leu-Pro-Ala-Gly-Gly-Gly-Thr-Val-
Leu-Thr-Lys-Met-Tyr-Pro-Arg-Gly-
Asn-His-Trp-Ala-Val-Gly-His-Leu-Met-NH₂.

The present invention also relates to salt forms of the peptides of Formula I. The peptides of Formula I as described herein are either sufficiently acidic or sufficiently basic to react with any of a number of inorganic bases, and inorganic and organic acids, to form a salt.

Acids commonly employed to form acid addition salts include inorganic acids such as hydrochloric acid, hydrobromic acid, hydroiodic acid, sulfuric acid, phosphoric acid, and the like, as well as organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, formate, isobutyrate, caproate, heptanoate, propiolate, oxalate, malonate, succinate, suberate, sebacate, fumarate, maleate, phthalate, sulfonate, phenylacetate, phenylpropionate, phenylbutyrate, citrate, lactate, glycolate, tartrate, methanesulfonate, propanesulfonate, naphthalene-1-sulfonate, naphthalene-2-sulfonate, mandelate, and the like.

Base addition salts include those derived from inorganic bases such as ammonium, alkali and alkaline earth metal hydroxides, carbonates, bicarbonates, and the like. Such bases thus include sodium hydroxide, potassium hydroxide, ammonium hydroxide, potassium carbonate, and the like.

Salt forms of the peptides of Formula I as described herein are particularly preferred. It is understood that the peptides of the present invention, when used for therapeutic purposes, may also be in the form of a salt. The salt, however, must be a pharmaceutically acceptable salt.

The present invention also relates to pharmaceutical compositions comprising a peptide of Formula I as described herein, in combination with a pharmaceutically acceptable carrier, diluent, or excipient. Such pharmaceutical compositions are prepared in a manner well known in the pharmaceutical art, and are administered individually or in combination with other therapeutic agents, preferably via parenteral routes. Particularly preferred routes include intramuscular and subcutaneous administration.

Parenteral daily dosages are in the range from about 1 mcg/kg to about 100 mcg/kg of body weight, although lower or higher dosages may be administered. The required dosage will depend upon the severity of the condition of the patient and upon such criteria as the patient's height, weight, sex, age, and medical history.

In preparing the compositions of the present invention, the active ingredient, which comprises at least one peptide of Formula I as described herein, is usually mixed with an excipient or diluted with an excipient. When an excipient is used as a diluent, it may be a solid, semi-solid, or liquid material, which acts as a vehicle, carrier, or medium for the active ingredient. Some examples of suitable excipients include lactose, dextrose, sucrose, trehalose, sorbitol, mannitol, starches, gum acacia, calcium silicate, microcrystalline cellulose, polyvinylpyrrolidone, cellulose, water, syrup, and methyl cellulose. The formulations can additionally comprise lubricating agents such as talc, magnesium stearate and mineral oil, wetting agents, emulsifying and suspending agents, preserving agents such as methyl- and propylhydroxybenzoates, as well as sweetening agents or flavoring agents. The compositions of the present invention can be formulated so as to provide quick, sustained or delayed release of the active ingredient following administration to the patient, following procedures well known in the art.

The compositions are preferably formulated in a unit dosage form with each dosage normally comprising from about 1 μg to about 10 mg of the active ingredient. The term “unit dosage form” refers to physically discrete units suitable as unitary dosages for human subjects and other mammals; each unit containing a predetermined quantity of active ingredient calculated to produce the desired therapeutic effect optionally in association with one or more suitable pharmaceutical excipients.

Additional pharmaceutical methods can be employed to control the duration of action. Controlled release preparations are obtained by the use of polymers, complexing or absorbing a peptide of Formula I as defined herein. The controlled release is obtained by selecting appropriate macromolecules (for example, polyesters, polyamino acids, polyvinylpyrrolidone, ethylenevinyl acetate, methylcellulose, carboxymethylcellulose, and protamine sulfate) as well as the concentration of the macromolecules, in addition to the methods of incorporation. Such teachings are disclosed in Remington's Pharmaceutical Sciences (16^{th} ed. 83: 1438-1497, Mack Publishing Company, Easten, Pa. 1980).

Another possible pharmaceutical method providing controlled release is to incorporate a peptide of Formula I as described herein, into particles of a polymeric material such as polyesters, polyamino acids, hydrogels, poly (lactic acid) or ethylene vinylacetate copolymers.

Given the sequence information disclosed herein and considering the state of the art in solid phase protein synthesis, the above-described peptides and peptide metabolites can be obtained via chemical synthesis. The principles of solid phase chemical synthesis of polypeptides are well known in the art ([1]. Dugas, H., Penney, C.; Bioorganic Chemistry (1981) Springer-Verlag, New York, pgs. 54-92; [2]. Merrifield, J. M., Chem. Soc., 85:2149 (1962), [3] Stewart and Young, Solid Phase Peptide Synthesis, pp. 24-66, Freeman (San Francisco, 1969).

The present invention is illustrated in further detail by the following non-limiting examples.

### EXPERIMENTAL

1. Abbreviations

DMF: N,N-Dimethylformamide; TFA: Trifluoroacetic acid; DIEA: Diisopropylethylamine; BOP: Benzotriazole-1-yl-oxy-tris (dimethylamino) phosphonium hexafluorophosphate; HPLC: High Performance Liquid Chromatography; MALDI-MS: Matrix Assisted Laser Desorption/Ionisation Mass Spectrometry; BHA.HCl: Benzhydrylamine resin hydrochloride salt; t-Bu: t-Butoxy; Pbf: 2,2,4,6,7-Pentamethyldihydrobenzofurane-5-sulfonyl; Boc: t-Butoxycarbonyl; Trt: Trityl; and Fmoc: Fluorenylmethoxycarbonyl.

2. Chemical Synthesis of the Peptides of Formula I

The pharmacophore “X”, which is a the acyl portion of the corresponding carboxylic acid “X—OH”, is anchored to amino groups such as those found at the N-terminus of peptides. The anchoring is preferably performed on solid phase support (Merrifield R. B. 1963, J. Am. Chem. Soc. 1963, 85, 2149 and J. Am. Chem. Soc. 1964, 86, 304) using benzotriazole-1-yl-oxy-tris (dimethylamino) phosphonium hexafluorophosphate (B. Castro et al., 1975, Tett. Lett., Vol. 14: 1219). The preferred working temperature ranges from about 20 to about 60° C. The anchoring reaction time, in the case of the more hydrophobic moieties, varies inversely with temperature, and varies from about 0.1 to 24 hours.

The synthesis steps were carried out by solid-phase methodology using a manual peptide synthesizer or an automatic peptide synthesizer following the Fmoc strategy. The BHA resin was used as the starting material. The coupling of the amino acids was done in DMF with 3 equivalents of amino acids, using 3 equivalents of BOP (Benzotriazole-1-yl-oxy-tris (dimethylamino) phosphonium hexafluorophosphate) as the coupling agent, and using 6 equivalents of DIEA as the nucleophilic agent. The coupling time was fixed at 60 minutes. Deprotection of the Fmoc protected N-terminus was performed using 20% piperidine/DMF. All the coupling reactions were monitored by a Kaiser test.

Final cleavage of side chain protecting groups and removal of the resin-bound peptide was performed using the following mixture: TFA, ethanedithiol, thioanisole, triisopropylsilane, water, phenol (90:2:2:2:2:2). A final concentration of 20 ml of cleavage cocktail per gram of dried peptide was used to cleave the peptide from the resin. The cleavage reaction was performed at room temperature for 2 hours. The free peptide, now in solution in the TFA cocktail, was then filtered on a coarse fritted disk funnel. The resin was then washed 3 times with pure TFA. The peptide/TFA mixture was evaporated under vacuum on a rotary evaporator, precipitated and washed with ether prior to its dissolution in water and freeze drying to eliminate the remaining traces of solvent and scavengers.

The peptides were purified by reverse-phase HPLC and analyzed using analytical HPLC and MS Maldi-TOF.

3. Coupling of the Pharmacophore “X”

The coupling of the acyl portion “X” of the corresponding carboxylic acid “X—OH” to the N-terminus of the resin-bound peptide was conducted under the same conditions as those of the Fmoc-amino acids. The corresponding carboxylic acid derivatives of “X” are commercially available or prepared using standard procedures known to one skilled in the art. In the following examples GLP-1 (9-36) amide is used as

4. Synthesis of 6-phenylhexanoyl-GLP-1 (9-36) NH₂ (214)

6-Phenylhexanoyl-GLP-1 (9-36) NH₂ was produced by solid phase peptide chemistry on a Symphony Multiplex Peptide Synthesizer (Rainin Instrument Co., Inc.) using BHA resin (0.44 mmol/g) as the starting material. The coupling of the amino acids was done in DMF with 3 equivalents of amino acids, using 3 equivalents of BOP as the coupling agent, and 6 equivalents of N-methylmorpholine as the nucleophilic agent (100 mmol scale). The coupling time was fixed at 60 minutes. Deprotection of the Fmoc protected N-terminus was performed using 20% piperidine/DMF. Amino acids with reactive side chains were protected as follows: Arg(Pbf); Lys(Boc); Trp(Boc); Glu(t-Bu); Tyr(t-Bu); Ser(t-Bu); Asp(Ot-Bu); Thr(t-But); Gln(Trt); His(Trt).

Residues were sequentially connected from the C-terminal towards the N-terminal end with a series of coupling and deprotection cycles. A coupling cycle consisted of the activated amino acid undergoing nucleophilic substitution by the free primary amine of the previously coupled amino acid. Deprotection involved the removal of the N-terminal blocking group Fmoc with 20% piperidine/DMF.

Once the peptide sequence was completed, the X moiety at the N-terminus of the GLP-1 (9-36), in this case the 6-phenylhexanoyl, was introduced using the corresponding carboxylic acid 6-phenylhexanoic acid, using the same conditions as those used for the Fmoc-amino acids. The peptide was then cleaved using a TFA cocktail (90% TFA, 2% ethanedithiol, 2% thioanisole, 2% triisopropylsilane, 2% water, 2% phenol) over a period of 2 hours, followed by precipitation using dry-ice cold Et₂O. The crude peptide was than purified by preparative reverse-phase HPLC, and analyzed by analytical HPLC and MS (Maldi-TOF).

A number of peptides were synthesized as previously described for the synthesis of 6-phenylhexanoyl-GLP-1 (9-36) NH₂, and are illustrated in Table 5.

TABLE 5
Peptides synthesized as described in Example 4.

| Com- | |
|---|---|
| pound | |
| No | Structure |
| 254 | 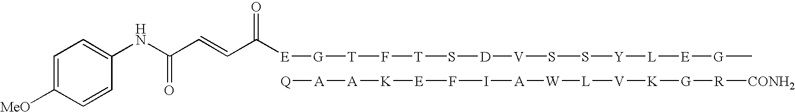 |
| 255 | 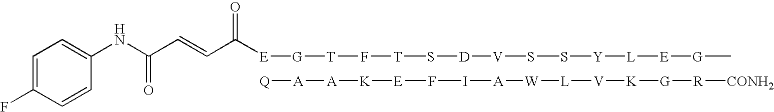 |
| 256 | 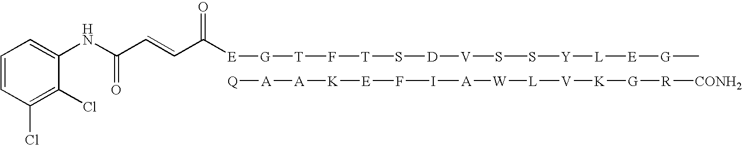 |
| 257 | 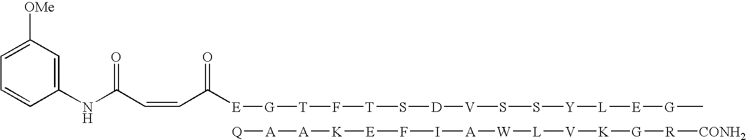 |
| 214 | 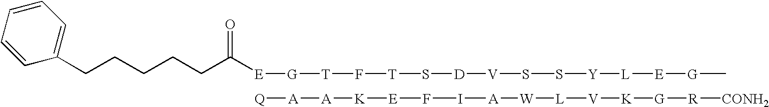 |
| 215 | 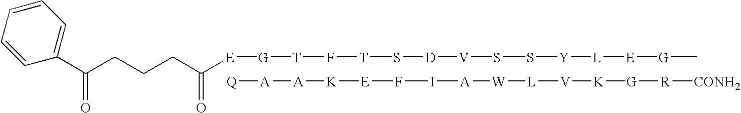 |
| 216 | 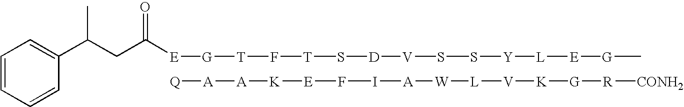 |
| 217 | 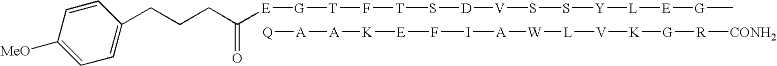 |
| 218 | 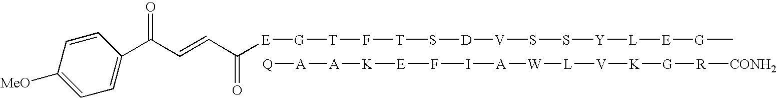 |
| 219 | 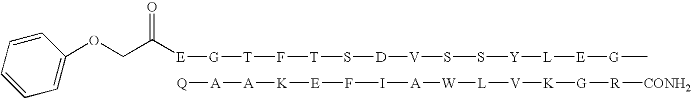 |
| 220 | 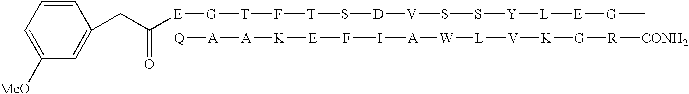 |
| 221 | 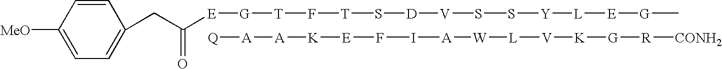 |
| 222 | 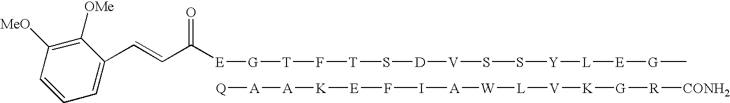 |
| 223 | 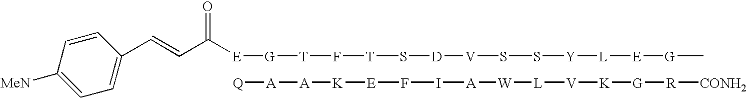 |
| 224 | 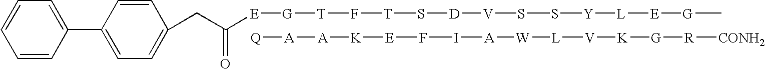 |
| 225 | 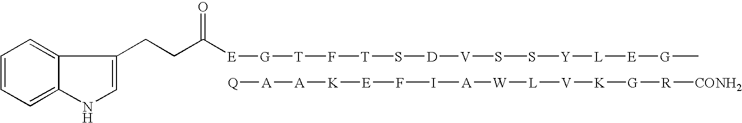 |
| 226 | 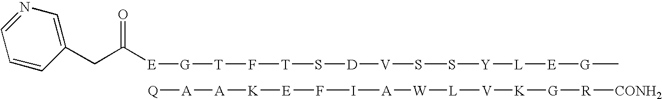 |
| 227 | 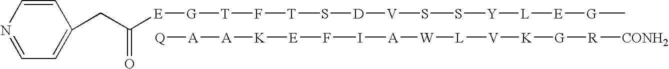 |
| 242 | 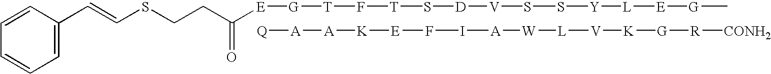 |
| 243 | 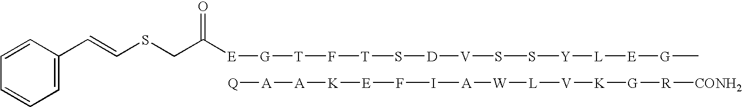 |
| 244 | 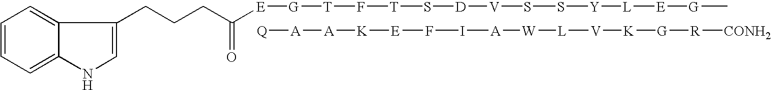 |
| 245 | 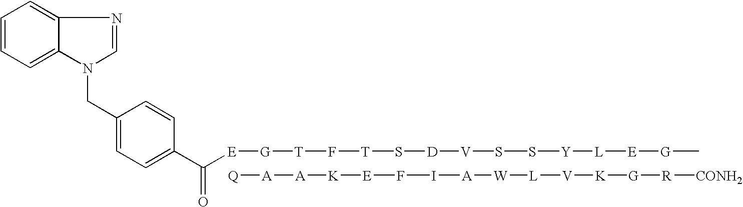 |
| 246 | 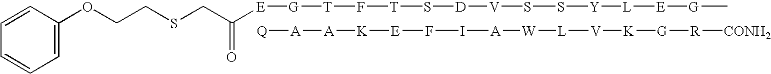 |
| 258 | 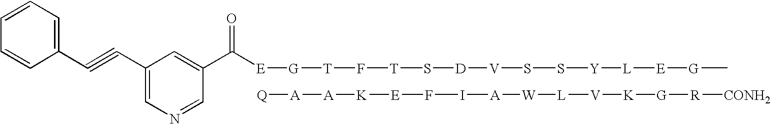 |
| 259 | 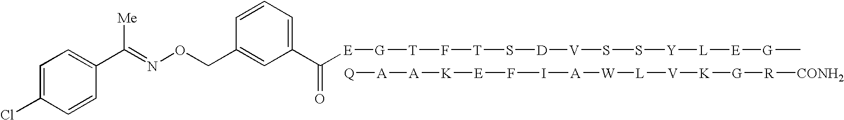 |
| 247 | 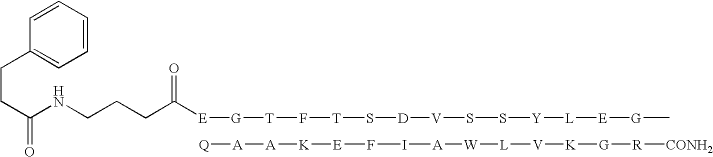 |
| 248 | 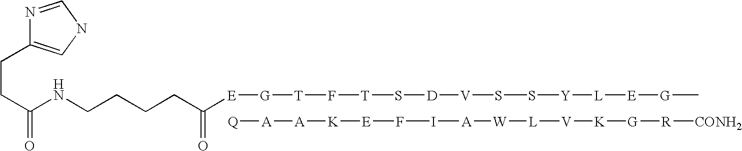 |
| 249 | 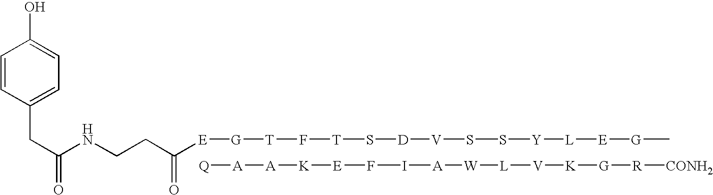 |
| 250 | 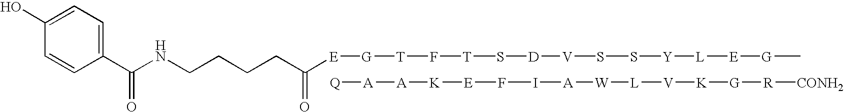 |
| 251 | 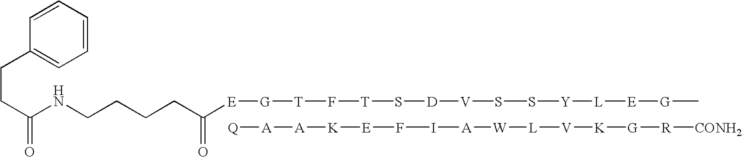 |
| 252 | 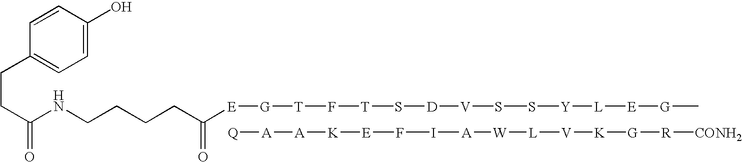 |
| 253 | 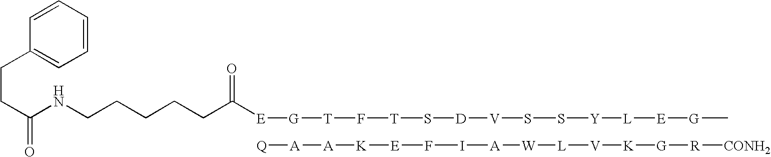 |

5. Synthesis of 3-(4-methoxyphenethylamino)-3-oxopriopionic Acid (31)

The synthesis of the carboxylic acid derivative of pharmacophore 17 (see Table 4) is depicted in Scheme 1. Methyl 3-chloro-oxopropionate (28) was reacted with 4-methoxyphenethylamine (29) in dichloromethane at 0° C. to give the desired methyl 3-(4-methoxyphenethylamine)-3-oxopriopionate (30) in 93.4% yield. Hydrolysis of the ester (30) with alcoholic NaOH afforded the desired acid 31 in 86% yield.

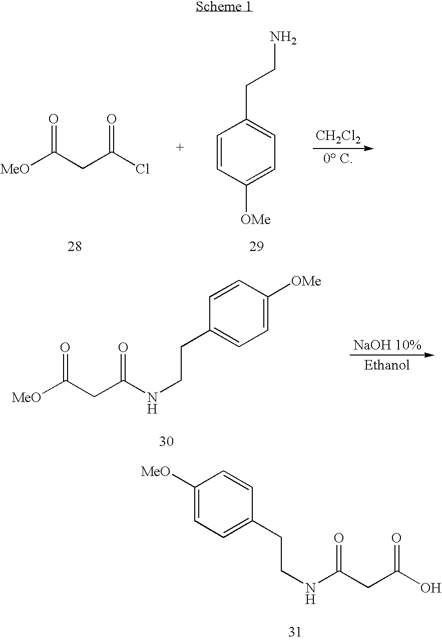

To a solution of methyl 3-chloro-3-oxopriopionate (28) (2.00 g, 14.6 mmol) in dichloromethane (40 ml) at 0° C., was added dropwise 4-methoxyphenethylamine (29) (3.32 g, 21.9 mmol) and the reaction allowed to stir for 1 hour at room temperature. Water (50 ml) was added and the reaction mixture was extracted with dichloromethane (3×50 ml). The extracts were combined and washed twice with saturated aqueous NaHCO₃ and aqueous 10% HCl. The extracts were then dried on MgSO₄, filtered and concentrated under reduced pressure to afford methyl 3-(4-methoxyphenethylamine)-3-oxopriopionate (30) as a yellow solid (3.43 g) in 93.4% yield.

Methyl 3-(4-methoxyphenethylamine)-3-oxopriopionate (30) (1.37 g) in ethanol (10 ml) and aqueous 10% NaOH (10 ml), was stirred at room temperature for 1 hour. The reaction mixture was evaporated to dryness and the residue re-dissolved in water, acidified with aqueous 10% HCl, and extracted with chloroform. The combined organic phases were subsequently dried over MgSO₄, filtered and concentrated under pressure to give 3-(4-methoxyphenethylamine)-3-oxopriopionic acid (31) as a white yellowish solid (1.12 g) in 86.8% yield.

5.1 Synthesis of 4-methoxyphenetylamine-mGly-GLP-1 (9-36) Amide (288)

The synthesis of 288 was carried out using a procedure essentially identical to the procedure used for preparing 214.

4-Methoxyphenetylamine-mGly-GLP-1 (9-36) amide (288) was prepared by solid phase peptide chemistry on a Symphony Multiplex Peptide Synthesizer (Rainin Instrument Co. Inc.) using BHA resin (0.44 mmol/g) as the starting material. The coupling of the amino acids was done in DMF using 3 equivalents of amino acids, 3 equivalents of BOP as the coupling agent, and 6 equivalents of N-methylmorpholine as the nucleophilic agent (100 mmol scale). The coupling time was fixed at 60 minutes. Deprotection of the Fmoc protected the N-terminus was performed using 20% piperidine/DMF. Amino acids with reactive side chains were protected as follows: Arg(Pbf); Lys(Boc); Trp(Boc); Glu(t-Bu); Tyr(t-Bu); Ser(t-Bu); Asp(t-Bu); Thr(t-Bu); Gln(Trt).

Residues were sequentially connected from the C-terminal towards the N-terminal end with a series of coupling and deprotection cycles. A coupling cycle consisted of the activated amino acid undergoing nucleophilic substitution by the free primary amine of the previously coupled amino acid. Deprotection involved the removal of the N-terminal blocking group Fmoc with 20% piperidine/DMF.

Once the peptide sequence was completed, the X moiety at the N-terminus of GLP-1 (9-36), in this case the acyl portion of the acid 31, was introduced using the same conditions as those used for the Fmoc-amino acids. The peptide was then cleaved using a TFA cocktail (90% TFA, 2% ethanedithiol, 2% thioanisole, 2% triisopropylsilane, 2% water, 2% phenol) over a period of 2 hours, followed by precipitation using ether. The crude peptide was than purified by preparative reverse- and phase HPLC, and analyzed by analytical HPLC and MS (Maldi-TOF).

The glucagon and GRF analogs 361, 280, and 358 were synthesized as described above.

TABLE 5

| Compound | |
|---|---|
| No | Structure |
| 287 | 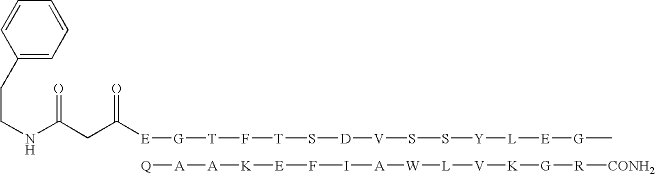 |
| 288 | 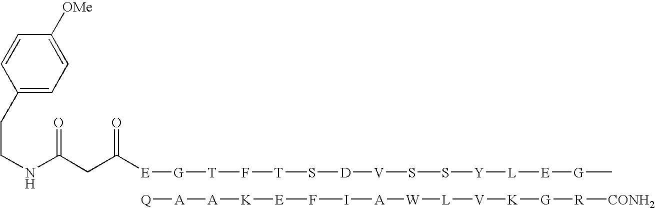 |
| 289 | 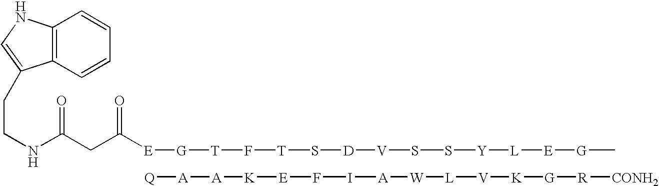 |
| 290 | 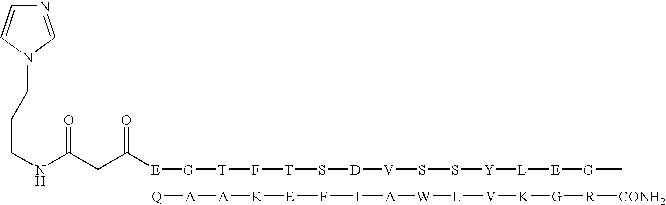 |
| 293 | 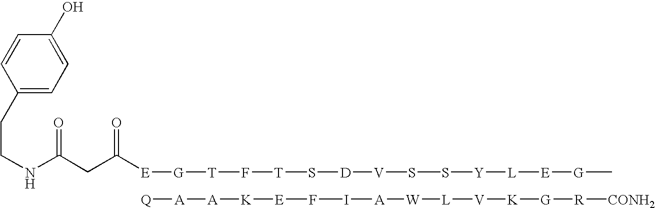 |
| 281 | 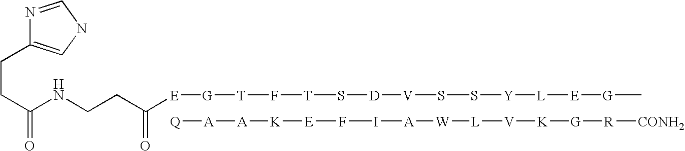 |
| 282 | 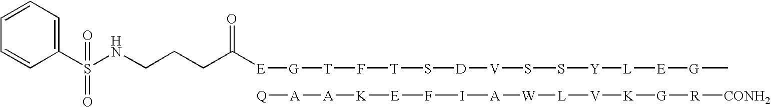 |
| 283 | 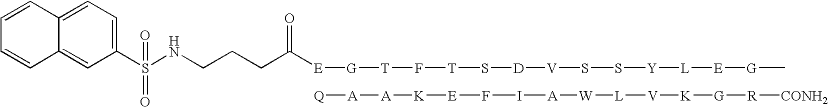 |
| 284 | 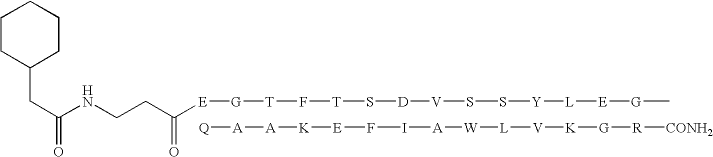 |
| 291 | 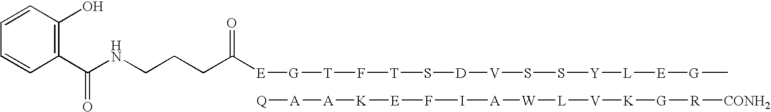 |
| 292 | 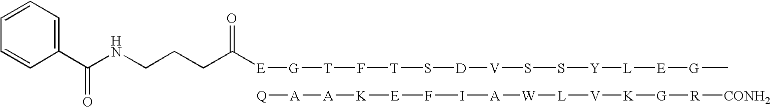 |
| 294 | 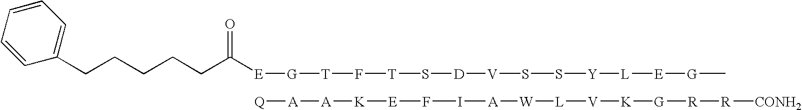 |
| 297 | 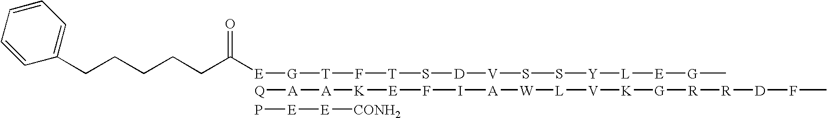 |
| 357 | Q—G—T—F—T—S—D—Y—S—K—Y—L—D—S—R—R—A—Q—D—F—V—Q—W—L—M—N—T— |
| | CONH₂ |
| | (Glucagon (3-29) amide) |
| Native | H—S—Q—G—T—F—T—S—D—Y—S—K—Y—L—D—S—R—R—A—Q—D—F—V—Q—W—L—M— |
| Glucagon | N—T—CONH₂ |
| | (Glucagon (1-29) amide) |
| 361 | 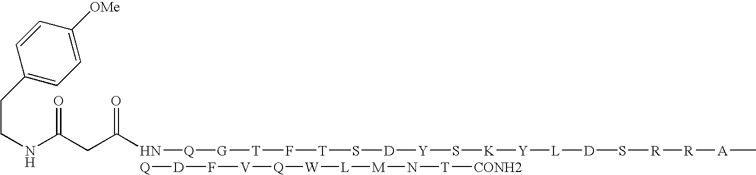 (4-Methoxyphenethylamine-mGly-Glucagon (3-29) amide) |
| 277 | E—G—T—F—T—S—D—V—S—S—Y—L—E—G—Q—A—A—K—E—F—I—A—W—L—V—K—G—R— |
| | CONH2 |
| | (GLP-1 (9-36) amide) |
| 356 | D—A—I—F—T—N—S—Y—R—K—V—L—G—Q—L—S—A—R—K—L—L—Q—D—I—M—S—R—CONH2 |
| | (GRF (3-29) amide) |
| 280 | 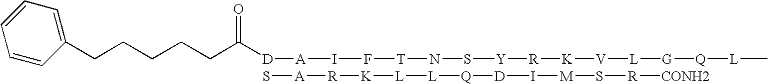 |
| 358 | 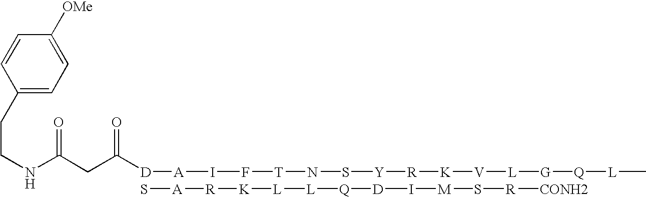 (4-Methoxyphenethylamine-mGly-GRF-1 (3-29) amide) |

6. Second Messenger (cAMP) Synthesis in Rat Insulinoma Cells (RINm5F) Produced by GLP-1 Analogues

RINm5F cells (ATCC # CRL-2058) were grown in ATCC recommended media and conditions. Cells (50 000 cells/well) were seeded and grown to confluence in 96-well plates (White Costar™ plate with clear bottom) in 100 μl medium. Stock solutions (1 mM) of GLP-1 and analogues were made in water containing 0.1% BSA. Aliquots of stock solutions were frozen at −20° C. Dilutions of peptides were made in HBBS (118 mM NaCl, 4.6 mM KCl, 1 mM CaCl₂, 10 mM D-Glucose, 20 mM Hepes, pH 7.2) containing 0.5 mM isobutylmethyl xanthine (IBMX) and used within 30 min.

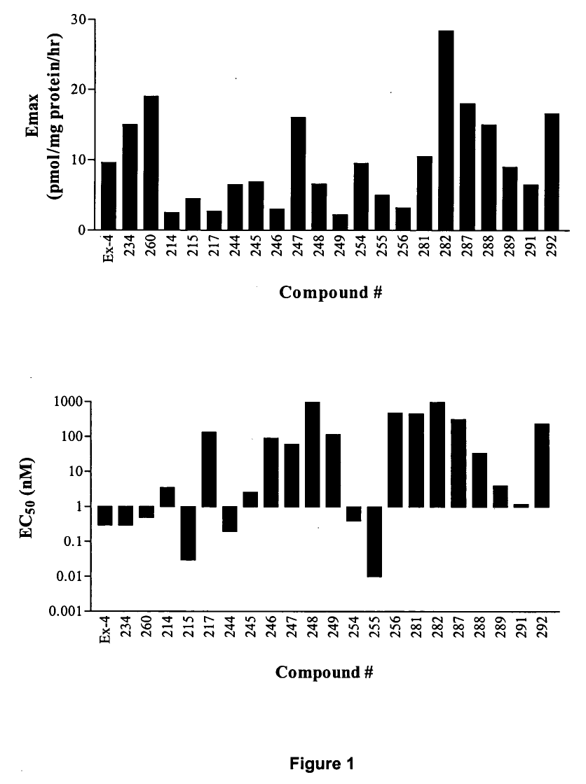

Cells were washed once with HBBS containing 0.5 mM IBMX and then pre-incubated in 90 μl HBBS/0.5 mM IBMX at 37° C. for 10 minutes. After pre-incubation, 10 μl of 100 nM GLP-1 analogs were added to wells and incubated for an additional 40 minutes. At the end of incubation, the supernatant was aspirated and cells are assayed directly for cAMP levels using a commercial kit (HitHunter™ EFC cAMP Chemiluminescence Assay kit for adherent cells; Applied Biosystems). Chemiluminescence in the wells was determined in a TopCount™ Scintillation and Luminescence counter (Packard). To measure protein, cells in four more wells were trypsinized, washed once with PBS and re-suspended in 100 μl PBS. The protein concentration was then quantified using a commercial reagent kit (Coomassie™ Blue, Pierce). Data were expressed as pmol cAMP/mg protein and transformed to percent increment over cAMP levels in vehicle-treated cells. Results are presented in FIG. 1, Several compounds were observed as having comparable Eₘₐₓ and EC₅₀ values to Exendin-4 (EX-4), 234 (GLP-1 [7-36]amide) and 260 (Gly8 GLP-1 [7-36] amide).

7. Insulin Secretion Stimulated by GLP-1 Analogues in Response to Intraperitoneal Glucose Tolerance Test (IPGTT)

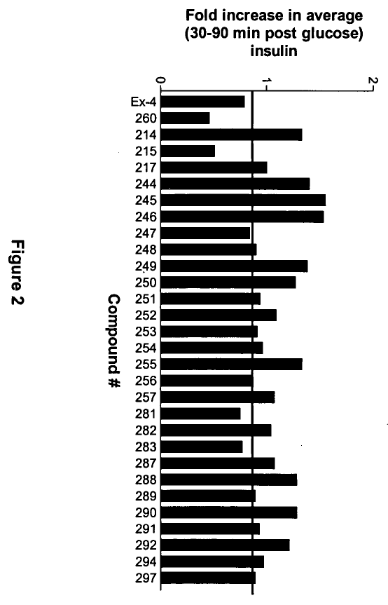

Sprague-Dawley rats (300-350 g) that fasted overnight, were injected with 1 g/kg glucose in 2 ml volume (over 15-20 sec) and blood glucose levels were determined at 30, 60 and 90 min using a portable glucometer (Lifescan). The drugs (10 μg/rat) were dissolved in saline and injected into the femoral vein 5 min before the injection of glucose. Thus “0” time represents insulin levels after drug administration but before glucose injection. Plasma insulin levels were determined by using an radioimmunoassay kit (Linco Research). Insulin levels were calculated in ng/ml. Results are presented in FIG. 2, which shows the insulin secretion stimulated by GLP-1 analogues of the present invention in response to the intraperitoneal glucose tolerance test (IPGTT). Insulin levels (30, 60 and 90 minutes following glucose challenge) are averaged. Taking the average insulin level of 234 (GLP-1 [7-36] amide) and 260 (Gly8 GLP-1 [7-36] amide) as 1.0, the fold increase in insulin produced by the compounds are calculated and presented as a bar graph. The horizontal line represents the average insulin level produced by 234 and 260. The compounds that produced average insulin levels at or above the line are considered to be equally or more potent than compounds 234 and 260.

8. Effects of Glucagon and Analogues on Freshly Isolated Hepatocytes cAMP Production

Hepatocytes Preparation

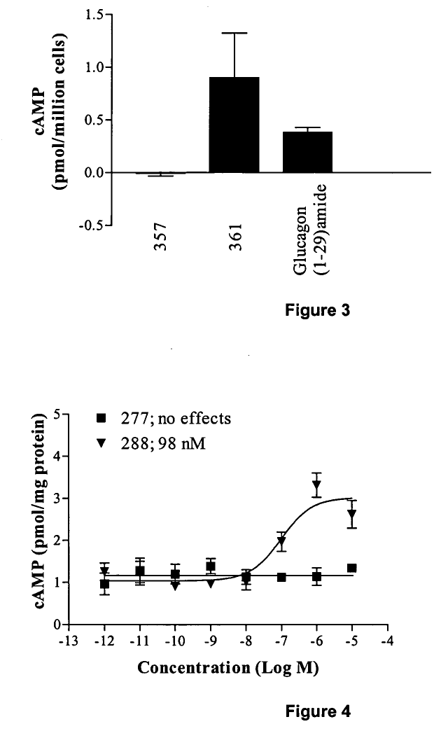

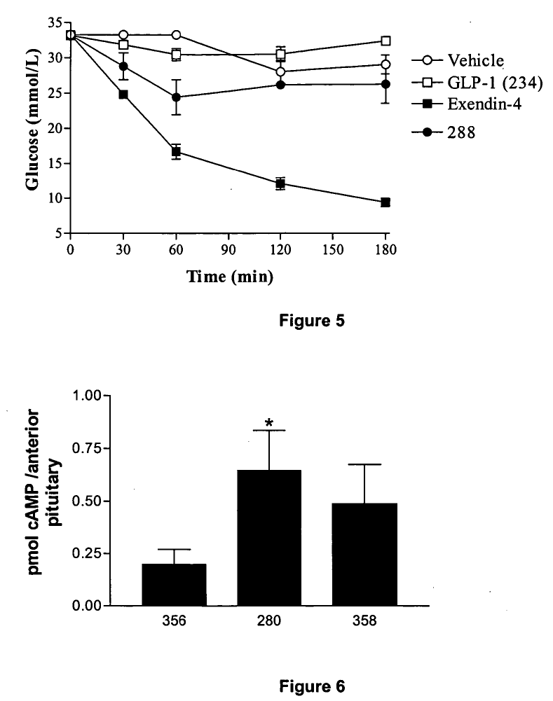

All experimental procedures were performed under isoflurane (2.5%) anesthesia according to an experimental protocol approved by Ste-Justine Hospital (Montreal) animal care committee. Briefly, an incision was made across the abdomen to reveal the liver and allow access to the superior vena cava. The animal was perfused through the heart to remove a maximal amount of blood from the liver (acquire a light brown color). A catheter (PE-90) was then inserted in the portal vein and the liver further perfused to eliminate any trace of blood. The hepatic artery was also cannulated (PE-50) and perfused. The liver was then carefully removed from the abdominal cavity and placed into a 250 ml beaker. Digesting HEPES buffer containing 9650U collagenase and 20U elastase at 37° C. was placed into the beaker and circulated in a closed loop via the catheters for 10 minutes at maximal speed. The buffer was replaced with a fresh solution of collagenase and elastase and perfusion continues for 10 additional minutes. The liver was transferred to a new beaker, buffer was added without collagenase or elastase and the hepatocytes dissociated by mechanical means (the peritoneum is opened and removed with scissors and tweezers and the liver agitated lightly for a few seconds) until pasty in appearance. The cells were filtrated with a tea strainer; the vascular tree and cell heaps remaining on the strainer. The cells are centrifuged at 52G for 3 minutes, resuspended and washed two more times. This gives 120 to 160 million live cells from 1 liver (300 g rat). As shown in FIG. 3, replacement of His-Ser dipeptide by a synthetic mimic in compound 361 elicited significantly higher intracellular cAMP levels as compared to analogue 357 lacking the His-Ser dipeptide (negligible response) or glucagon itself. Furthermore, FIG. 6 shows cAMP production stimulated by GRF analogues (10⁻⁶ M) in whole anterior pituitary culture. Analogue 358 produced a significantly greater cAMP response than analogue 356. For comparison, a DPPIV resistant analogue of GRF (analogue 280) was shown.

cAMP Stimulation Assay

Stimulation studies were performed at a concentration of 1 million cells per tube; 5 minutes of pre-treatment with 0.1 mM IBMX, with or without glucagon agonists (10⁻⁷M) compared to treatment with glucagon (10⁻⁷M). Reactions were stopped on ice and stored at −80° C. prior to ETOH extraction. The cell pellets were thawed by adding 500 μl of 70% ETOH, vortexing for a few seconds and incubating at 37° C. for 10 min. The tubes were centrifuged at 13,000×g for 10 min at 4° C. and the supernatants lyophilized in a speed-vac. The cAMP levels in the tubes were determined using a radioimmunoassay kit (Amersham DPC kit). The data are expressed as pmol cAMP/million cells.

9. Dose Response Curves of GLP-1 Analogues on cAMP Production in RINm5F Cells

Preparation of RINm5F Cells

RINm5F cells (ATCC # CRL-2058) were grown according to the manufacturer's specifications. Cells from −90% confluent flasks were trypsinized and counted. 20 000 cells/well were seeded in 96-well plate (White Costar plate with clear bottom) in 100 μl media. Cells were grown four days past confluence before being using for experiments.

cAMP Stimulation Assay

Stock solutions of agents tested were prepared in DDH₂O+0.1% BSA at a concentration of 1 mM (correcting for peptide purity and peptide content when available) immediately prior to the beginning of the assay. From the stock solutions, 2× dilutions (2×10⁻¹² M to 2×10⁻⁵ M) were made in RPMI medium containing 0.5 mM IBMX. Cell culture media was gently removed from wells. Cells were then washed once with RPMI containing 0.5 mM IBMX and then pre-incubated in 100 μl RPMI/0.5 mM IBMX at 37° C. for 10 minutes. After pre-incubation, 100 μl of each 2× dilutions were added to wells in triplicates and incubated at 37° C. for 40 minutes. At the end of incubation, the supernatant was collected and assayed for cAMP using a radioimmunoassay kit (DPC). Radioactive counts were transformed into cAMP amounts using a standard curve. cAMP values were corrected for total protein content in the corresponding wells determined by Coomassie blue assay. cAMP values were expressed as mean±SEM in pmol/mg protein (FIG. 2). Dose responses of cAMP were fitted to a sigmoidal curve model (fixed slope) using GraphPad Prism 3.02. cAMP data was also represented in some figures as percentage increase over basal (basal being 100%). As shown in FIG. 4, analogue 288, increased CAMP production.

Although the present invention has been described hereinabove by way of preferred embodiments thereof, it can be modified without departing from the spirit and the nature of the subject invention as defined in the appended claims.

1. A peptide of Formula I, or a pharmaceutically acceptable salt thereof:
X—P Formula I
wherein:
P is a DPPIV peptide metabolite of regulatory peptides obtained by cleavage of the two N-terminal amino acids; and
X is defined by Formula II: 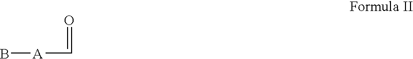
wherein:
A is selected from the group consisting of C₁-C₁₀ alkylene, C₂-C₁₀ alkenylene, C₂-C₁₀ alkynylene, C₁-C₁₀ heteroalkylene, C₂-C₁₀ heteroalkenylene, C₂-C₁₀ heteroalkynylene and phenyl; and
B is selected from the group consisting of aryl, substituted aryl, heteroaryl, substituted heteroaryl and C₃-C₇ cycloalkyl.

2. The peptide of claim 1, wherein X is selected from the group consisting of:

| | |
|---|---|
| 1 | 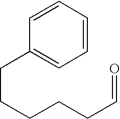 |
| 2 | 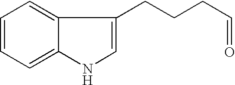 |
| 3 | 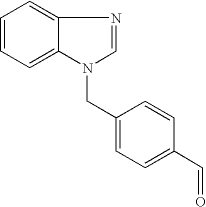 |
| 4 | 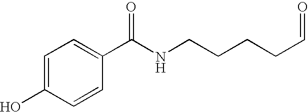 |
| 5 | 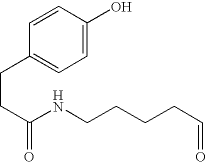 |
| 6 | 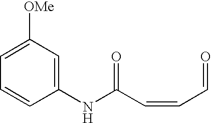 |
| 7 | 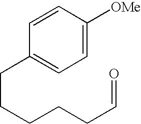 |
| 8 | 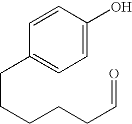 |
| 9 | 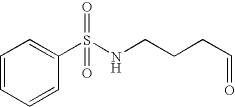 |
| 10 | 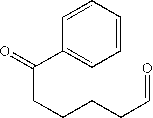 |
| 11 | 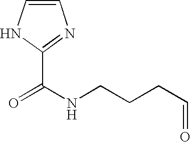 |
| 12 | 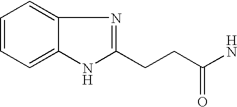 |
| 13 | 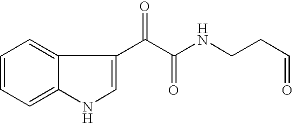 |
| 14 | 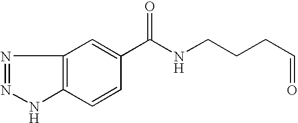 |
| 15 | 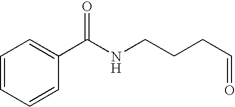 |
| 16 | 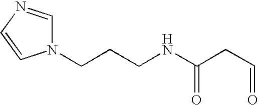 |
| 17 | 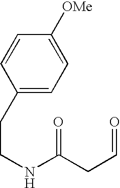 |
| 18 | 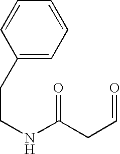 |
| 19 | 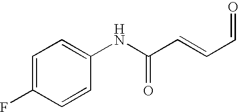 |
| 20 | 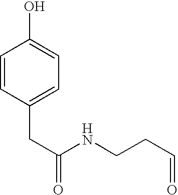 |
| 21 | 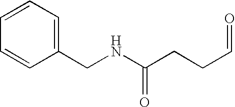 |
| 22 | 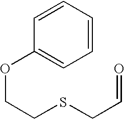 |
| 23 | 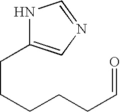 |
| 24 | 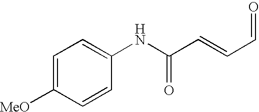 |
| 25 | 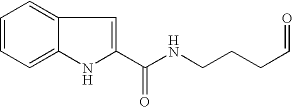 |
| 26 | 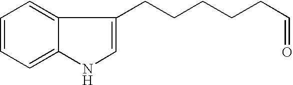 |
| 27 | 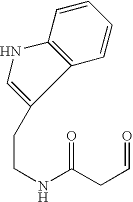 |
| 28 | 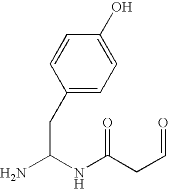 |
| 29 | 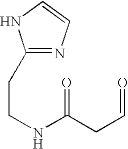 |
| 30 | 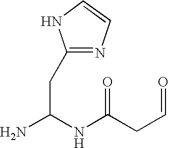 |
| 31 | 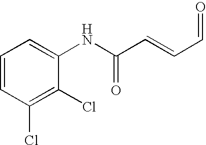 |
| 32 | 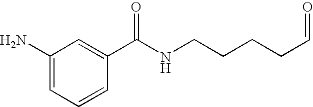 |
| 33 | 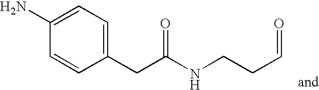 |
| 34 | 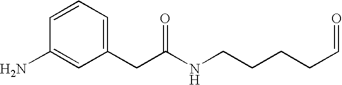 |

3. The peptide of claim 1, wherein the regulatory peptides are selected from the group consisting of Glucagon Like Peptide-1 (GLP-1), Glucagon Like Peptide-2 (GLP-2), Growth Hormone Releasing Hormone (GHRH), Vasoactive Intestinal Peptide (VIP), Glucose-dependent Insulinotropic Peptide (GIP), Glucagon, Neuropeptide Y, Peptide YY, Gastrin Releasing Peptide (GRP) and salts thereof.

4. The peptide of claim 1, wherein P is Glucagon Like Peptide-1 (GLP-1) metabolite (9-44) NH₂ having the following sequence:
Glu-Gly-Thr-Phe-Thr-Ser-Asp-
Val-Ser-Ser-Tyr-Leu-Glu-Gly-Gln-
Ala-Ala-Lys-Glu-Phe-Ile-Ala-Trp-Leu-Val-Lys-Gly-
Arg-Gly-Arg-Arg-Asp-Phe-Pro-Glu-
Glu-NH₂;
and wherein X is as defined in claim 2.

5. The peptide of claim 1, wherein P is Glucagon Like Peptide-1 (GLP-1) metabolite (9-39) amide having the following sequence:
Glu-Gly-Thr-Phe-Thr-Ser-Asp-
Val-Ser-Ser-Tyr-Leu-Glu-Gly-Gln-
Ala-Ala-Lys-Glu-Phe-Ile-Ala-Trp-Leu-Val-Lys-Gly-
Arg-Gly-Arg-Arg-NH₂;
and wherein X is as defined in claim 2.

6. The peptide of claim 1, wherein P is Glucagon Like Peptide-1 (GLP-1) metabolite (9-37) having the following sequence:
Glu-Gly-Thr-Phe-Thr-Ser-Asp-Val-Ser-
Ser-Tyr-Leu-Glu-Gly-Gln-Ala-Ala-
Lys-Glu-Phe-Ile-Ala-Trp-Leu-Val-Lys-Gly-Arg-Gly-OH;
and wherein X is as defined in claim 2.

7. The peptide of claim 1, wherein P is Glucagon Like Peptide-1 (GLP-1) metabolite (9-36) amide having the following sequence:
Glu-Gly-Thr-Phe-Thr-Ser-Asp-
Val-Ser-Ser-Tyr-Leu-Glu-Gly-Gln-
Ala-Ala-Lys-Glu-Phe-Ile-Ala-Trp-Leu-Val-Lys-Gly-
Arg-NH₂;
and wherein X is as defined in claim 2.

8. The peptide of claim 1, wherein P is Glucagon Like Peptide-2 (GLP-2) metabolite (3-34) having the following sequence:
Asp-Gly-Ser-Phe-Ser-Asp-Glu-
Met-Asn-Thr-Ile-Leu-Asp-Asn-Leu-
Ala-Ala-Arg-Asp-Phe-Ile-Asn-Trp-Leu-Ile-Gln-Thr-
Lys-Ile-Thr-Asp-Arg;
and wherein X is as defined in claim 2.

9. The peptide of claim 1, wherein P is Glucagon Like Peptide-2 (GLP-2) metabolite (3-33) having the following sequence:
Asp-Gly-Ser-Phe-Ser-Asp-Glu-
Met-Asn-Thr-Ile-Leu-Asp-Asn-Leu-
Ala-Ala-Arg-Asp-Phe-Ile-Asn-Trp-Leu-Ile-Gln-Thr-
Lys-Ile-Thr-Asp;
and wherein X is as defined in claim 2.

10. The peptide of claim 1, wherein P is Growth Hormone Releasing Factor (GRF) metabolite (3-44) NH₂ having the following sequence:
Asp-Ala-Ile-Phe-Thr-Asn-Ser-
Tyr-Arg-Lys-Val-Leu-Gly-Gln-Leu-
Ser-Ala-Arg-Lys-Leu-Leu-Gln-Asp-Ile-Met-Ser-Arg-
Gln-Gln-Gly-Glu-Ser-Asn-Gln-Glu-
Arg-Gly-Ala-Arg-Ala-Arg-Leu-NH₂;
and wherein X is as defined in claim 2.

11. The peptide of claim 1, wherein P is Growth Hormone Releasing Factor (GRF) metabolite (3-29) NH₂ having the following sequence:
Asp-Ala-Ile-Phe-Thr-Asn-Ser-
Tyr-Arg-Lys-Val-Leu-Gly-Gln-Leu-
Ser-Ala-Arg-Lys-Leu-Leu-Gln-Asp-Ile-Met-Ser-Arg-
NH₂;
and wherein X is as defined in claim 2.

12. The peptide of claim 1, wherein P is Vasoactive Intestinal Peptide (VIP) metabolite (3-28) NH₂ having the following sequence:
Asp-Ala-Val-Phe-Thr-Asp-Asn-
Tyr-Thr-Arg-Leu-Arg-Lys-Gln-Met-
Ala-Val-Lys-Lys-Tyr-Leu-Asn-Ser-Ile-Leu-Asn-NH₂;
and wherein X is as defined in claim 2.

13. The peptide of claim 1, wherein P is Glucose-Dependent Insulinotropic Peptide (GIP) metabolite (3-42) NH₂ having the following sequence:
Glu-Gly-Thr-Phe-Ile-Ser-Asp-
Tyr-Ser-Ile-Ala-Met-Asp-Lys-Ile-His-
Gln-Gln-Asp-Phe-Val-Asn-Trp-Leu-Leu-Ala-Gln-Lys-
Gly-Lys-Lys-Asn-Asp-Trp-Lys-His-
Asn-Ile-Thr-Gln;
and wherein X is as defined in claim 2.

14. The peptide of claim 1, wherein P is Glucose-Dependent Insulinotropic Peptide (GIP) metabolite (3-30) NH₂ having the following sequence:
Glu-Gly-Thr-Phe-Ile-
Ser-Asp-Tyr-Ser-Ile-Ala-Met-Asp-Lys-Ile-His-
Gln-Gln-Asp-Phe-Val-Asn-Trp-Leu-Leu-Ala-Gln-Lys;
and wherein X is as defined in claim 2.

15. The peptide of claim 1, wherein P is Glucagon metabolite (3-29) NH₂ having the following sequence:
Gln-Gly-Thr-Phe-Thr-Ser-Asp-Tyr-Ser-Lys-Tyr-
Leu-Asp-Ser-Arg-Arg-Ala-Gln-
Asp-Phe-Val-Gln-Trp-Leu-Met-Asn-Thr;
and wherein X is as defined in claim 2.

16. The peptide of claim 1, wherein P is neuropeptide Y metabolite (3-36) NH₂ having the following sequence:
Ser-Lys-Pro-Asp-Asn-Pro-Gly-
Glu-Asp-Ala-Pro-Ala-Glu-Asp-Met-
Ala-Arg-Tyr-Tyr-Ser-Ala-Leu-Arg-His-Tyr-Ile-Asn-
Leu-Ile-Thr-Arg-Gln-Arg-Tyr;
and wherein X is as defined in claim 2.

17. The peptide of claim 1, wherein P is peptide YY metabolite (3-29) NH₂ having the sequence:
Ile-Lys-Pro-Glu-Ala-Pro-Gly-
Glu-Asp-Ala-Ser-Pro-Glu-Glu-Leu-
Asn-Arg-Tyr-Tyr-Ala-Ser-Leu-Arg-His-Tyr-Leu-Asn-
Leu-Val-Thr-Arg-Gln-Arg-Tyr-NH₂;
and wherein X is as defined in claim 2.

18. The peptide of claim 1, wherein P is Gastrin Releasing Peptide (GRP) metabolite (3-27) NH₂ having the sequence:
Leu-Pro-Ala-Gly-Gly-Gly-Thr-
Val-Leu-Thr-Lys-Met-Tyr-Pro-Arg-
Gly-Asn-His-Trp-Ala-Val-Gly-His-Leu-Met-NH₂;
and wherein X is as defined in claim 2.

19. A composition comprising a therapeutically effective amount of the peptide of claim 1 in association with at least one pharmaceutically acceptable carrier, diluent or excipient.

20. The composition of claim 19, wherein said therapeutically effective amount is from about 1 mcg to about 10 mg.

21. A method for treating or preventing a disease or condition associated with a disorder of glucose metabolism comprising administering to a subject in need thereof, a therapeutically effective amount of a peptide selected from the group consisting of: the peptide of claim 4, the peptide of claim 5, the peptide of claim 6 and the peptide of claim 7.

22. The method of claim 21, wherein said therapeutically, effective amount is from about 1 mcg to about 10 mg.

23. The method of claim 22, wherein said disease or condition associated with a disorder of glucose metabolism is selected from the group consisting of diabetes mellitus of Type I or Type II and insulin resistance.

24. The method of claim 23, wherein said disease or condition is diabetes mellitus Type I or Type II.

25. The method of claim 23, wherein said disease or condition is insulin resistance.

26. The method of claim 21, wherein said disease or condition is a weight disorder or associated condition.

27. The method of claim 26, wherein said weight disorder or associated condition is selected from at least one of lowering weight, increasing satiety, post-prandially increasing plasma insulin levels, reducing blood glucose levels, and increasing pancreatic beta cell mass in said subject.

28. The method of claim 27, wherein said lowering weight is from about 1 to about 10 Kg.

29. The method of claim 27, wherein said increasing satiety is about 10%.

30. The method of claim 27, wherein said post-prandially increasing plasma insulin levels is about 10%.

31. The method of claim 27, wherein said reducing blood glucose levels is of the order of about 10%.

32. The method of claim 27, wherein said increasing pancreatic beta cell mass is of the order of about 10%.

33. The method of claim 21, wherein said peptide is administered to said subject through an administration route selected from the group consisting of subcutaneous, intravenous, transdermal, oral, bucal, and intranasal.

34. The method of claim 33, wherein said subject is human.

35. A composition comprising a prophylactically effective amount of the peptide of claim 1, in association with at least one constituent selected from the group consisting of pharmaceutically acceptable carriers, diluents and excipients.


SEQUENCE LISTING (52 sequences)

SEQ ID NO: 1 - protein, 11 aa, Artificial Sequence
  Description of Artificial Sequence  Synthtic Peptide
RPLPQEFFGLM

SEQ ID NO: 2 - protein, 5 aa, Artificial Sequence
  Description of Artificial Sequence  Synthtic Peptide
YPFPG

SEQ ID NO: 3 - protein, 4 aa, Artificial Sequence
  Description of Artificial Sequence  Synthtic Peptide
YPFF

SEQ ID NO: 4 - protein, 5 aa, Artificial Sequence
  Description of Artificial Sequence  Synthtic Peptide
VPDPR

SEQ ID NO: 5 - protein, 8 aa, Artificial Sequence
  Description of Artificial Sequence  Synthtic Peptide
YPSKPDNP

SEQ ID NO: 6 - protein, 8 aa, Artificial Sequence
  Description of Artificial Sequence  Synthtic Peptide
YPIKPEAP

SEQ ID NO: 7 - protein, 9 aa, Artificial Sequence
  Description of Artificial Sequence  Synthtic Peptide
SAKELRCQC

SEQ ID NO: 8 - protein, 10 aa, Artificial Sequence
  Description of Artificial Sequence  Synthtic Peptide
GPVSAVLTEL

SEQ ID NO: 9 - protein, 10 aa, Artificial Sequence
  Description of Artificial Sequence  Synthtic Peptide
EAEEDGDLQC

SEQ ID NO: 10 - protein, 11 aa, Artificial Sequence
  Description of Artificial Sequence  Synthtic Peptide
VPLSRTVRCTC

SEQ ID NO: 11 - protein, 11 aa, Artificial Sequence
  Description of Artificial Sequence  Synthtic Peptide
TPVVRKGRCSC

SEQ ID NO: 12 - protein, 11 aa, Artificial Sequence
  Description of Artificial Sequence  Synthtic Peptide
KPVSLSYRCPC

SEQ ID NO: 13 - protein, 11 aa, Artificial Sequence
  Description of Artificial Sequence  Synthtic Peptide
APLATELRCQC

SEQ ID NO: 14 - protein, 11 aa, Artificial Sequence
  Description of Artificial Sequence  Synthtic Peptide
FPMFKKGRCLC

SEQ ID NO: 15 - protein, 10 aa, Artificial Sequence
  Description of Artificial Sequence  Synthtic Peptide
SPYSSDTTPC

SEQ ID NO: 16 - protein, 11 aa, Artificial Sequence
  Description of Artificial Sequence  Synthtic Peptide
APLAADTPTAC

SEQ ID NO: 17 - protein, 11 aa, Artificial Sequence
  Description of Artificial Sequence  Synthtic Peptide
APMGSDPPTAC

SEQ ID NO: 18 - protein, 10 aa, Artificial Sequence
  Description of Artificial Sequence  Synthtic Peptide
QPDANAPVTC

SEQ ID NO: 19 - protein, 9 aa, Artificial Sequence
  Description of Artificial Sequence  Synthtic Peptide
QPSDVSPTC

SEQ ID NO: 20 - protein, 9 aa, Artificial Sequence
  Description of Artificial Sequence  Synthtic Peptide
QPVGNSTTC

SEQ ID NO: 21 - protein, 11 aa, Artificial Sequence
  Description of Artificial Sequence  Synthtic Peptide
QPDALDVPSTC

SEQ ID NO: 22 - protein, 9 aa, Artificial Sequence
  Description of Artificial Sequence  Synthtic Peptide
GPASVPTTC

SEQ ID NO: 23 - protein, 12 aa, Artificial Sequence
  Description of Artificial Sequence  Synthtic Peptide
GPYGANMEDSVC

SEQ ID NO: 24 - protein, 28 aa, Artificial Sequence
  MOD_RES (28): X = anything
EGTFTSDVSSYLEGQAAKEFAWLVKGRX

SEQ ID NO: 25 - protein, 28 aa, Artificial Sequence
  Description of Artificial Sequence  Synthtic Peptide
DGSFSDEMNTLDNLAARDFNWLQTKTDR

SEQ ID NO: 26 - protein, 40 aa, Artificial Sequence
  Description of Artificial Sequence  Synthtic Peptide
DAFTNSYRKVLGQLSARKLLQDMSRQQGESNQERGARARL

SEQ ID NO: 27 - protein, 25 aa, Artificial Sequence
  Description of Artificial Sequence  Synthtic Peptide
DAVFTDNYTRLRKQMAVKKYLNSLN

SEQ ID NO: 28 - protein, 36 aa, Artificial Sequence
  Description of Artificial Sequence  Synthtic Peptide
EGTFSDYSAMDKHQQDFVNWLLAQKGKKNDWKHNTQ

SEQ ID NO: 29 - protein, 27 aa, Artificial Sequence
  Description of Artificial Sequence  Synthtic Peptide
QGTFTSDYSKYLDSRRAQDFVQWLMNT

SEQ ID NO: 30 - protein, 32 aa, Artificial Sequence
  Description of Artificial Sequence  Synthtic Peptide
SKPDNPGEDAPAEDMARYYSALRHYNLTRQRY

SEQ ID NO: 31 - protein, 33 aa, Artificial Sequence
  Description of Artificial Sequence  Synthtic Peptide
KPEAPGEDASPEELNRYYASLRHYLNLVTRQRY

SEQ ID NO: 32 - protein, 25 aa, Artificial Sequence
  Description of Artificial Sequence  Synthtic Peptide
LPAGGGTVLTKMYPRGNHWAVGHLM

SEQ ID NO: 33 - protein, 36 aa, Artificial Sequence
  Description of Artificial Sequence  Synthtic Peptide
EGTFTSDVSSYLEGQAAKEFIAWLVKGRGRRDFPEE

SEQ ID NO: 34 - protein, 31 aa, Artificial Sequence
  Description of Artificial Sequence  Synthtic Peptide
EGTFTSDVSSYLEGQAAKEFIAWLVKGRGRR

SEQ ID NO: 35 - protein, 29 aa, Artificial Sequence
  Description of Artificial Sequence  Synthtic Peptide
EGTFTSDVSSYLEGQAAKEFIAWLVKGRG

SEQ ID NO: 36 - protein, 28 aa, Artificial Sequence
  Description of Artificial Sequence  Synthtic Peptide
EGTFTSDVSSYLEGQAAKEFIAWLVKGR

SEQ ID NO: 37 - protein, 32 aa, Artificial Sequence
  Description of Artificial Sequence  Synthtic Peptide
DGSFSDEMNTILDNLAARDFINWLIQTKITDR

SEQ ID NO: 38 - protein, 31 aa, Artificial Sequence
  Description of Artificial Sequence  Synthtic Peptide
DGSFSDEMNTILDNLAARDFINWLIQTKITD

SEQ ID NO: 39 - protein, 42 aa, Artificial Sequence
  Description of Artificial Sequence  Synthtic Peptide
DAIFTNSYRKVLGQLSARKLLQDIMSRQQGESNQERGARARL

SEQ ID NO: 40 - protein, 27 aa, Artificial Sequence
  Description of Artificial Sequence  Synthtic Peptide
DAIFTNSYRKVLGQLSARKLLQDIMSR

SEQ ID NO: 41 - protein, 26 aa, Artificial Sequence
  Description of Artificial Sequence  Synthtic Peptide
DAVFTDNYTRLRKQMAVKKYLNSILN

SEQ ID NO: 42 - protein, 40 aa, Artificial Sequence
  Description of Artificial Sequence  Synthtic Peptide
EGTFISDYSIAMDKIHQQDFVNWLLAQKGKKNDWKHNITQ

SEQ ID NO: 43 - protein, 28 aa, Artificial Sequence
  Description of Artificial Sequence  Synthtic Peptide
EGTFISDYSIAMDKIHQQDFVNWLLAQK

SEQ ID NO: 44 - protein, 27 aa, Artificial Sequence
  Description of Artificial Sequence  Synthtic Peptide
QGTFTSDYSKYLDSRRAQDFVQWLMNT

SEQ ID NO: 45 - protein, 34 aa, Artificial Sequence
  Description of Artificial Sequence  Synthtic Peptide
SKPDNPGEDAPAEDMARYYSALRHYINLITRQRY

SEQ ID NO: 46 - protein, 34 aa, Artificial Sequence
  Description of Artificial Sequence  Synthtic Peptide
IKPEAPGEDASPEELNRYYASLRHYLNLVTRQRY

SEQ ID NO: 47 - protein, 25 aa, Artificial Sequence
  Description of Artificial Sequence  Synthtic Peptide
LPAGGGTVLTKMYPRGNHWAVGHLM

SEQ ID NO: 48 - protein, 28 aa, Artificial Sequence
  Description of Artificial Sequence  Synthtic Peptide
EGTFTSDVSSYLEGQAAKEFIAWLVKGR

SEQ ID NO: 49 - protein, 31 aa, Artificial Sequence; identical to SEQ ID NO: 34
  Description of Artificial Sequence  Synthtic Peptide

SEQ ID NO: 50 - protein, 36 aa, Artificial Sequence; identical to SEQ ID NO: 33
  Description of Artificial Sequence  Synthtic Peptide

SEQ ID NO: 51 - protein, 27 aa, Artificial Sequence
  Description of Artificial Sequence  Synthtic Peptide
QGTFTSDYSKYLDSRRAQDFVQWLMNT

SEQ ID NO: 52 - protein, 29 aa, Artificial Sequence
  Description of Artificial Sequence  Synthtic Peptide
HSQGTFTSDYSKYLDSRRAQDFVQWLMNT